(19) United States
(12) Patent Application Publication
(10) Pub. No.: US 2025/0099348 A1
(43) Pub. Date: Mar. 27, 2025

(54) ORAL CARE COMPOSITION COMPRISING TREATED ARAGONITE CALCIUM CARBONATE

(71) Applicant: Visionaturolab Inc., Terrebonne (CA)

(72) Inventor: David HAUPTMAN, Blainville (CA)

(21) Appl. No.: 18/833,088

(22) PCT Filed: Jan. 26, 2023
(86) PCT No.: PCT/CA2023/050094

§ 371 Date: Jul. 25, 2024

Related U.S. Application Data
(60) Provisional application No. 63/303,186, filed on Jan. 26, 2022.

(51) Int. Cl. A61K 8/19; A61Q 11/00

(52) U.S. Cl. CPC A61K 8/19; A61Q 11/00; A61K 2800/40; A61K 2800/61; A61K 2800/651

(57) ABSTRACT

The present document describes an oral care composition comprising treated aragonite calcium carbonate (CaCO₃) particles having more than 95% (w/w) calcium carbonate content, a specific surface area (SSA) of about 2.70 to about 3.1 m²/g, for use as a first dental abrasive, a fluoride compound suitable to provide beneficial fluoride treatment to teeth; and a suitable carrier. The treated aragonite calcium carbonate particles have been effectively treated in mildly acidic condition to avoid reaction of fluoride from the fluoride compound and the treated aragonite calcium carbonate particles.

### CROSS-REFERENCE TO RELATED APPLICATIONS

This application claims priority of U.S. Provisional Patent Application No. 63/303,186 filed Jan. 26, 2022, the specification of which is hereby incorporated by reference in its entirety.

### BACKGROUND

### (a) Field

The subject matter disclosed generally relates to oral care compositions and uses thereof, and more specifically, the subject matter disclosed relates to oral care compositions comprising cuttlefish bone powder and uses thereof.

### (b) Related Prior Art

Bacterial plaque and calculus are major etiological factors in the initiation and progression of periodontal disease. Dental calculus is a mineralized plaque and because it is porous, it can absorb various toxic products and consequently serves as an ideal substrate for subgingival microbial colonization. Hence, calculus should be accurately detected and thoroughly removed for adequate periodontal therapy. Many techniques have been used to identify and remove calculus deposits that are present on the teeth surface. Maintenance of proper hygiene of the oral cavity is one of the most important measures to be undertaken to fight gingivitis. The selection of the proper toothpaste is the first step in the fight with oral disease. Brushing efficiently to remove dental plaque has always been a concern against tooth wear and gum recession. Brushing alone has little wearing effect and therefore, loss of enamel as a result of brushing is mainly the result of abrasives used in toothpastes. On the other hand, most of the cleaning action while brushing is linked to the abrasive materials in the toothpaste and therefore, their presence is essential for cleaning. Toothpastes that are available today in the market have different kinds of abrasives in their formula, such as calcite, calcite and aragonite, silicon dioxide, brushite, gibbsite, etc.

Moreover, toothpastes are commonly produced to serve multiple purposes simultaneously thus, possess a complex chemical composition. Ideally, the formulation of a toothpaste should be balanced to maintain maximum cleaning benefit while minimizing the abrasive damage to the teeth structure. Therefore, excessively abrasive materials can abrade the tooth surface away, resulting in undesirable tooth wear and sensitivity. Many factors define the degree of abrasivity of a given compound, including its hydration level; the size, hardness, shape, and concentration of the particulate components; source; purity; as well as the method it has been treated physically and chemically. Also, toothpastes may act as vehicles for antimicrobial agents that may have a preventive/therapeutic role in periodontal disease. The complex composition of toothpastes implies that it is necessary to ensure that the active ingredients are not inactivated in the process of production or delivery. For instance, calcium carbonate added to dentifrice binds to fluoride, rendering the latter ineffective as an anti-caries agent (Shen et al. “Bioavailable fluoride in calcium-containing dentifrices” Scientific Reports, (2021) 11:146). Therefore, the composition of toothpastes is critical for their effectiveness on oral health maintenance and safety for the oral cavity.

Tooth mineral is lost and gained in a continuous process of de- and re-mineralization. Caries (dental decay) is a disease of the hard tissues of the teeth caused by an imbalance in this process over time, where there is demineralization of tooth structure by organic acids formed from the interactions between cariogenic bacteria in dental plaque and fermentable carbohydrates (mainly sugars). The dental caries process is influenced by the susceptibility of the tooth surface, the bacterial profile, the quantity and quality of saliva and the presence of fluoride, which promotes remineralization and inhibits demineralization of the tooth structure. Aside from the pain arising from the dental carious lesions themselves, there is also the emotional distress of the disease and the potential consequences of medical intervention. Affected teeth cannot always be saved and may have to be extracted. There are also important financial implications for this disease with a substantial proportion of healthcare budgets being spent every year on treating caries. On a population basis, traditional treatment of oral disease (dental caries and periodontal diseases) is the fourth most expensive chronic disease to treat according to the World Health Organization (WHO).

Caries in permanent teeth was the most prevalent condition among all those evaluated in the Global Burden of Disease 2016 study, affecting 2.4 billion people; the estimated prevalence of caries in deciduous teeth was 486 million children worldwide (GBD 2016). Whilst in some areas of middle-income and high-income countries, there has been evidence of a reduction in the prevalence and severity of dental caries in recent decades, social inequalities in dental health exist, with many individuals and communities having a clinically significant burden of preventable dental disease. Levels of dental decay vary considerably between and within countries, but children in lower socio-economic status (SES) groups have higher caries levels than those in upper SES groups, and in high-income countries, the association between socio-economic position and caries might be stronger.

The present description assesses the efficacy of toothpastes containing a specific preparation of treated aragonite calcium carbonate (CaCO₃) as abrasive, that shows a lesser degree of reaction and interference with fluoride, providing a greater bioavailability.

### SUMMARY

It is against the above background that the present invention provides certain advantages and advancements over the prior art.

According to an embodiment, there is provided an oral care composition comprising:
treated aragonite calcium carbonate (CaCO₃) particles having more than 95% (w/w) calcium carbonate content, a specific surface area (SSA) of about 2.70 to about 3.1 m²/g, for use as a first dental abrasive;
a fluoride compound suitable to provide beneficial fluoride treatment to teeth; and
a suitable carrier.
   wherein said treated aragonite calcium carbonate particles have been effectively treated in mildly acidic condition to avoid reaction of fluoride from said fluoride compound and said treated aragonite calcium carbonate particles.

The treated aragonite calcium carbonate particles may have a particle size of from about 25 microns to about 70 microns.

The calcium carbonate content may be from about 95% to about 99.9% (w/w).

The particles may have a specific surface area of from about 2.80 m²/g to about 2.9 m²/g.

The particles may have a specific surface area of from about 2.9 m²/g.

The treated aragonite calcium carbonate (CaCO₃) particles may be from an aragonite of vegetal origin.

The aragonite of vegetal origin may be an oolitic aragonite.

The fluoride compound may be sodium fluoride (NaF), stannous fluoride (SnF₂), sodium monofluorophosphate (MFP), or combinations thereof.

The fluoride compound may provide a concentration of fluoride of from about 800 ppm to about 5000 ppm.

The fluoride compound may provide a concentration of fluoride of from about 1000 ppm to about 1500 ppm.

The treated aragonite calcium carbonate particles may be from about 0.100% to about 20% (w/w) of the composition.

The particles may have a crystallinity of about 24% to about 28%.

The particles may have a crystallinity of about 26%.

The oral composition may further comprise a second dental abrasive.

The second dental abrasive may be a colloidal calcium, a colloidal silica, a hydrated silica, a sodium bicarbonate (NaHCO₃), aluminum hydroxide (Al(OH)₃), calcium carbonate (CaCO₃), a calcium hydrogen phosphate (CaHPO₄·2H₂O), an anhydrous calcium hydrogen phosphate, a silica, a zeolites, and hydroxyapatite (Ca₅(PO₄)₃OH), or a combination thereof.

The second dental abrasive may be a sodium bicarbonate (NaHCO₃), a colloidal silica, or a combination thereof.

The second dental abrasive may be from about 0.100% to about 30% (w/w) of the composition.

The colloidal silica may be from about 0.100% to about 20% (w/w) of the composition.

The sodium bicarbonate (NaHCO₃) may be from about 0.02% to about 0.75% (w/w) of the composition.

The oral care composition may further comprise a thickening agent.

The oral care composition of claim 20, wherein said thickening agent is a natural gum obtained from seaweeds; a natural gum obtained from non-marine botanical resource, a natural gum produced by bacterial fermentation, a starch, a pectin, a carboxymethyl cellulose, a hydroxypropyl cellulose, a methyl cellulose, a gelatin, a silica, or a combination thereof.

The natural gums obtained from seaweeds may be chosen from agar (E406), alginic acid (E400), Sodium alginate (E401), potassium alginate, ammonium alginate, calcium alginate, carrageenan (E407), or a combination thereof.

The natural gum obtained from non-marine botanical resource may be chosen from acacia gum, gum arabic (E414), gum ghatti, gum tragacanth (E413), karaya gum (E416), guar gum (E412), locust bean gum (E410), beta-glucan, chicle gum, dammar gum, Glucomannan (E425), mastic gum, psyllium seed husks, spruce gum, tara gum (E417), or a combination thereof.

The natural gum produced by bacterial fermentation may be chosen from gellan gum (E418), Xanthan gum (E415), or a combination thereof.

The thickening agent may be from about 0.1% to about 66% (w/w) of the composition. The thickening agent may be about 0.5% (w/w) of the composition.

The oral care composition may further comprise a humectant.

The humectant may be propylene glycol, hexylene glycol, butylene glycol, glyceryl triacetate, neoagarobiose, a sugar polyol, a polymeric polyol, quillaia, lactic acid, urea, glycerin, aloe vera gel, MP Diol, an alpha hydroxy acid, and honey.

The sugar polyols may be chosen from glycerol, sorbitol, xylitol, maltitol, and a combination thereof.

The polymeric polyol may be polydextrose, polyethylene glycol, polypropylene glycol, poly(tetramethylene ether) glycol, and a combination thereof.

The alpha hydroxy acid may be lactic acid.

The humectant may be glycerol, xylitol, sorbitol, or a combination thereof.

The humectant may be from about 2% to about 45% (w/w) of the composition.

The oral care composition may further comprise an emulsifier.

The emulsifier may be lecithin, a vegetal pulp powder, a sodium citrate and citric acid, or a combination thereof.

The vegetal pulp powder may be chosen from citrus pulp powder, baobab pulp powder, mango pulp powder, tomato pulp powder, pumpkin pulp powder, guava pulp powder, papaya pulp powder and beet pulp powder, or a combination thereof.

The sodium citrate may be trisodium citrate.

The emulsifier may be from about 1% to about 10% (w/w) of the composition.

The oral composition may further comprise a surfactant.

The surfactant may be chosen from sodium lauryl sulfate, ammonium lauryl sulfate, sodium N-lauryl sarcosinate, sodium lauryl sulfoacetate, or a combination thereof.

The surfactant may be from about 0.5% to about 3% (w/w) of the composition.

The oral composition may further comprise a pH regulator.

The pH regulator may be chosen from citric acid and its derivatives, phosphoric acid and its derivatives, trisodium phosphate, sodium citrate, lactic acid, bicarbonic acid, or a combination thereof.

The pH regulator may be from about 0.1% to about 0.75% (w/w) of the composition.

The oral composition may further comprise a preservative.

The preservative may be chosen from a sorbitan sesquioleate derivative, sodium benzoate, benzoic acid, a eucalyptus extract, potassium sorbate, or a combination thereof.

The preservative may be from about 0.2% to about 2% (w/w) of the composition.

The oral composition may further comprise a solvent.

The solvent may be chosen from water, ethanol, isopropanol, sorbitol and glycerol.

The solvent may be from about 40% to about 99% (w/w) of said composition.

The oral composition may further comprise an antimicrobial agent.

The antimicrobial agent may be chosen from a natural essential oil, an antimicrobial phenolic compound, or a combination thereof.

The natural essential oil may be chosen from oils of anise, lemon oil, orange oil, oregano, rosemary oil, wintergreen oil, thyme oil, lavender oil, clove oil, hops, tea tree oil, citronella oil, wheat oil, barley oil, lemongrass oil, cedar leaf oil, cedar wood oil, cinnamon oil, fleagrass oil, geranium oil, sandalwood oil, violet oil, cranberry oil, eucalyptus oil, vervain oil, peppermint oil, gum benzoin, basil oil, fennel oil, fir oil, balsam oil, menthol, ocmea origanum oil, Hydastis carradensis oil, Berberidaceae daceae oil, Ratanhiae and Curcuma longa oil, sesame oil, macadamia nut oil, evening primrose oil, Spanish sage oil, Spanish rosemary oil, coriander oil, thyme oil, pimento berries oil, rose oil, bergamot oil, rosewood oil, chamomile oil, sage oil, clary sage oil, cypress oil, sea fennel oil, frankincense oil, ginger oil, grapefruit oil, jasmine oil, juniper oil, lime oil, mandarin oil, marjoram oil, myrrh oil, neroli oil, patchouli oil, pepper oil, black pepper oil, petitgrain oil, pine oil, rose otto oil, spearmint oil, spikenard oil, vetiver oil, or ylang ylang.

The antimicrobial phenolic compound may be chosen from carvacrol, thymol, eugenol, eucalyptol, menthol, or a combination thereof.

The antimicrobial agent may be from about 0.01% to about 10% (w/w) of the composition.

The oral composition may further comprise flavoring.

The flavoring may comprise menthol, a mint essential oil, or combinations thereof.

The treated aragonite calcium carbonate (CaCO₃) particles may be free of chitin.

The use of an oral composition of the present invention, for oral hygiene.

According to another embodiment, there is provided a use of an oral composition of the present invention, for removal of calculus, for prevention of calculus formation, or a combination thereof.

According to another embodiment, there is provided a method of cleaning an oral cavity comprising applying the oral composition of the present invention to an oral cavity.

According to another embodiment, there is provided a method of preventing formation of, or of removing calculus in an oral cavity comprising applying the oral composition of the present invention to an oral cavity.

According to another embodiment, there is provided an oral composition according to the present invention for use in oral hygiene.

According to another embodiment, there is provided an oral composition according to the present invention for use in the removal of calculus, for use in the prevention of calculus formation, or a combination thereof.

According to another embodiment, there is provided a method for the preparation of treated aragonite calcium carbonate (CaCO₃) particles having a reduced or inhibited reaction with fluoride from a fluoride compound suitable to provide beneficial fluoride treatment to teeth, the method comprising:
a) grinding an aragonite to obtain a coarse aragonite powder;
b) sieving said coarse aragonite powder to obtain a first ground aragonite powder having particle size of from about 60 microns to about 75 microns;
c) treating said ground aragonite with a mild acid at a pH of about 4.5 to 5.5, at a temperature sufficient and for a time sufficient to demineralize said first ground aragonite and obtain a demineralized ground aragonite;
d) washing said demineralized ground aragonite until a neutral pH is reached;
e) drying said demineralized ground aragonite, to obtain treated aragonite calcium carbonate (CaCO₃) particles having more than 95% (w/w) calcium carbonate content, and a specific surface area (SSA) of about 2.70 to about 3.1 m²/g.

The mild acid may be ammonium chloride or ammonium acetate, preferably ammonium chloride.

The concentration of ammonium chloride is from about 0.1 M to about 10 M, preferably 1.87M (10% w/v).

The step c) may be at a pH of about 4.5. The step c) may be at a pH of about 4.9. The step c) may be at a pH of about 4.86.

The step c) may be at a temperature from about 65° C. to about 75° C.

The step d) may be in distilled water.

The step e) may be at about 200° C. to about 220° C. The step e) may be at about 200° C.

The step e) may be for about 30 min to about 60 min. The step e) may be for about 55 min.

The following terms are defined below.

The term “oolitic aragonite” is intended to mean calcium carbonate mineral, aragonite, with an egg-like shape (“oolitic” from the Ancient Greek word [symbol] óv for “egg”) and sand grain size. This type or aragonite mineral typically forms in tropical waters through precipitation, sedimentation, and microbial activity, and is indicative of high energy environments. Oolitic aragonite forms in high-salinity waters that are turbulent, shallow, and warm. The oolittic aragonite starts to form around a nucleus of calcium carbonate, such as a peloid, shell fragment, or foraminifer. The nucleus is coated with a thin layer of crystalline carbonate to form the cortex of the ooid. Compared to other types of sand formation that involve the weathering and erosion of a larger rock by turbulent waters, oolitic aragonite sand is created by dissolved calcium carbonate joining with the cortex or nucleus of the ooid. The dissolved calcium carbonate in seawater continues to stick to the cortex and is combined with the high velocity water which creates the smooth, granular shape resulting in the aragonite composed ooid. Biomineralization involving microbial organic matter likely also plays an important role in ooid formation.

The term “reduced reaction rate” is intended refer to the reaction rate or rate of reaction of a chemical reaction, which is the speed at which a chemical reaction takes place, defined as proportional to the increase in the concentration of a product per unit time and to the decrease in the concentration of a reactant per unit time. Generically, reaction rates can vary dramatically. With respect to the present invention, the reaction rate is that of the fluoride compound present in the oral care composition of the present invention with the calcium present in the composition in the form of aragonite. Without wishing to be bound by theory, the treated oolitic aragonite particles of the present invention have been shown to have reduced reaction with the fluoride compound, such that after aging of the oral care composition, the amount of bioavailable fluoride is higher than in an oral care composition where the aragonite particles are different.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A illustrates the measures of particle size of CB, TCB, synthetic calcium carbonate (CaCO₃) and treated oolitic aragonite.

FIG. 2 illustrates is a FTIR characterization of treated oolitic aragonite versus treated cuttlebone aragonite (TCB). The treated oolitic aragonite spectrum shows the absence of the C═O band at 1653 cm⁻¹ corresponding to Chitin.

FIG. 3 shows XRD characterization of treated oolitic aragonite versus treated cuttlebone aragonite (TCB). The diffractogram (right) of the TCB showed wider band width indicates smaller particle size.

FIG. 4A illustrates a Scanning Electron Micrograph (SEM) of cuttlefish bone (CB) powder.

FIG. 4B illustrates a SEM of treated cuttlefish bone (CB) powder.

FIG. 4C illustrates a SEM of synthetic calcium carbonate (CaCO₃) powder.

FIG. 4D illustrates a SEM of cuttlefish bone (CB) powder of the sample shown in 4A, at a higher magnification.

FIG. 4E illustrates a SEM of treated cuttlefish bone (TCB) powder of the sample shown in 4B, at a higher magnification.

FIG. 4F illustrates a SEM of synthetic calcium carbonate (CaCO₃) powder of the sample shown in 4C, at a higher magnification.

FIG. 4G illustrates a SEM of treated oolitic aragonite powder of the present invention.

FIG. 4H illustrates a SEM of treated oolitic aragonite powder of the present invention of the sample shown in 4G, at a higher magnification.

FIG. 4I illustrates a SEM of treated oolitic aragonite powder of the present invention of the sample shown in 4G, at a higher magnification.

FIG. 4J illustrates a SEM of treated oolitic aragonite powder of the present invention of the sample shown in 4G, at a higher magnification.

FIG. 4K illustrates Energy-dispersive X-ray spectroscopy (EDX) analysis comparing treated cuttlefish bone (TCB) powder and treated oolitic aragonite.

FIG. 5 illustrates (top left) a SEM of Calculus surface Before reaction with treated oolitic aragonite; (top right) a SEM of Calculus surface After reaction with treated oolitic aragonite, and (bottom) EDX analysis comparing calculus before and after treatment with treated oolitic aragonite.

FIG. 6 illustrates the BET specific surface area of CB, TCB, synthetic calcium carbonate (CaCO₃) and treated oolitic aragonite (identified as Aragonite).

FIG. 7 illustrates the Abrasion depth of TCB, synthetic calcium carbonate (CaCO₃), and treated oolitic aragonite (identified as Aragonite) slurries on enamel, dentin and calculus.

FIG. 8 illustrates the determined mineral phase abundance of calculus samples. Amorphous calcium phosphate (ACP), α-tricalcium phosphate (α-TCP), dicalcium phosphate dihydrate (DCPD, in the form of brushite), hydroxyapatite (HA), and aragonite CaCO₃.

FIG. 9A illustrates the correlation of amounts of non-Apatitic calcium phosphate (CaP) vs Apatitic calcium phosphate (CaP) [i.e., the total of hydroxyapatite (HA) and crystalline hydroxyapatite (CHA)] in the calculus samples.

FIG. 9B illustrates the correlation of amounts of α-TCP vs. DCPD in the calculus samples.

FIG. 9C illustrates the correlation of amounts of α-TCP vs. Aragonite in the calculus samples.

FIG. 9D illustrates the correlation of amounts of DCPD vs. Aragonite in the calculus samples.

FIG. 9E illustrates the correlation of amounts of DCPD vs. HA in the calculus samples.

FIG. 9F illustrates the correlation of amounts of α-TCP vs. HA in the calculus samples.

FIG. 9G illustrates the correlation of amounts of HA vs. Aragonite in the calculus samples.

FIG. 10A illustrates the reactivity of equal molar ratio of calcite CaCO₃ and brushite dicalcium phosphate dihydrate (DCPD) measured with FTIR analysis in H₂O (top) or not (bottom).

FIG. 10B illustrates the reactivity of equal molar ratio of TCB and brushite dicalcium phosphate dihydrate (DCPD) measured with FTIR analysis in H₂O (top) or not (bottom).

FIG. 10C illustrates the subtraction of the measurements from FIG. 10A, which shows that based on the magnitude of the value F, no reaction is taking place.

FIG. 10D illustrates the subtraction of the measurements from FIG. 10B, which shows that based on the magnitude of the value F, a reaction between the two compounds is taking place.

FIG. 10E illustrates the reactivity of equal molar ratio of treated oolitic aragonite (ARG) and brushite dicalcium phosphate dihydrate (DCPD) measured with FTIR analysis in H₂O (top) or not (bottom).

FIG. 10F illustrates the reactivity of equal molar ratio of calcite CaCO₃ and β-tricalcium phosphate (BTCP) measured with FTIR analysis in H₂O (top) or not (bottom).

FIG. 10G illustrates the subtraction of the measurements from FIG. 10E, which shows that based on the magnitude of the value F, a reaction between the two compounds is taking place.

FIG. 10H illustrates the subtraction of the measurements from FIG. 10F, which shows that based on the magnitude of the value F, only a weak reaction between the two compounds is taking place.

FIG. 10I illustrates the reactivity of equal molar ratio of treated oolitic aragonite (ARG) and β-tricalcium phosphate (BTCP) measured with FTIR analysis in H₂O (top) or not (bottom).

FIG. 10J illustrates the reactivity of equal molar ratio of TCB and β-tricalcium phosphate (BTCP) measured with FTIR analysis in H₂O (top) or not (bottom)

FIG. 10K illustrates the subtraction of the measurements from FIG. 10I, which shows that based on the magnitude of the value F, only a weak reaction between the two compounds is taking place.

FIG. 10L illustrates the subtraction of the measurements from FIG. 10J, which shows that based on the magnitude of the value F, only a weak reaction between the two compounds is taking place.

FIG. 11A illustrates the reactivity of equal molar ratio of treated oolitic aragonite and calculus measured with FTIR analysis in H₂O (top) or not (bottom).

FIG. 11B illustrates the subtraction of the measurements from FIG. 11A, which shows that based on the magnitude of the value F, a reaction between the two compounds is taking place

FIG. 11C illustrates the reactivity of equal molar ratio of calcite CaCO₃ and calculus measured with FTIR analysis in H₂O (top) or not (bottom).

FIG. 11D illustrates the subtraction of the measurements from FIG. 11C, which shows that based on the magnitude of the value F, no, or only a weak reaction between the two compounds is taking place.

FIG. 12A illustrates FTIR spectra of calcite, aragonite, and treated aragonite powders before and after 14 days of incubation in saturated calcium phosphate solution (*: PO₄³⁻ group).

FIG. 12B illustrates calcium ion concentration in the supernatant.

FIG. 12C illustrates phosphorus ion concentration in the supernatant.

FIG. 13 illustrates the anticalculus action of pyrophosphate which inhibits calculus formation by inhibiting calcium phosphate deposition in plaque.

FIG. 14 is a Schematic diagram representing the reaction between aragonite or treated aragonite and dental calculus, according to an embodiment of the present invention.

It will be noted that throughout the appended drawings, like features are identified by like reference numerals.

### DETAILED DESCRIPTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

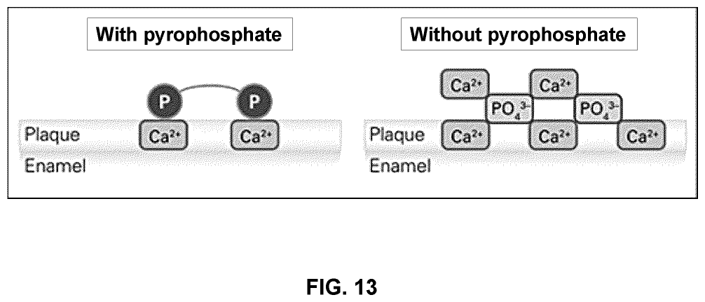

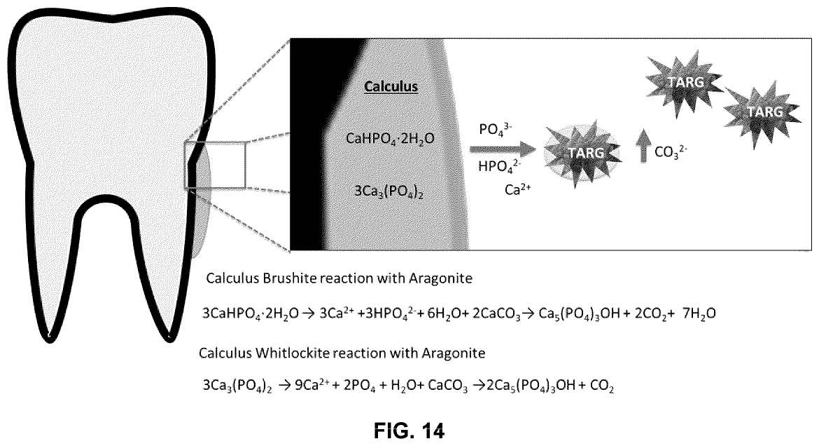

Dental calculus is mineralized plaque and because it is porous, it can absorb various toxic chemicals, food debris and bacteria that can damage the periodontal tissues. Hence, calculus removal is critical for maintaining adequate periodontal health. Therefore, there has been substantial interest in the development and implementation of approaches that will ease the calculus removal process. Currently, the only feasible method for removing dental calculus is mechanical removal by scaling in dental clinics. Toothpastes may also include a variety of abrasives, including calcite, silicon dioxide, brushite, and gibbsite, which are essential for cleaning, however, they may also damage enamel and dentin while removing calculus. The toothpastes contain carboxylates and pyrophosphate they used at low concentrations in toothpastes for demineralization of calculus but when using these at high concentration it may dissolve enamel. Dental calculus is made of calcium phosphate crystals, which are created when calcium and phosphate bind to form calcium phosphate crystals. Other ways for removing and preventing calculus may be assisted by brushing with pyrophosphate-containing toothpaste, such as Crest® Tartar Protection, which adheres to the tooth's surface and prevents calculus crystals from forming or developing by preventing amorphous calcium phosphate from crystallizing into hydroxyapatite. Pyrophosphates work by building a soluble complex with the calcium in plaque to prevent the crystal formation (deposition) of the minerals in plaque on teeth (See e.g., FIG. 13).

Despite being chelating agents with low toxicity, pyrophosphates, however, prevent hydroxyapatite from crystallizing in bones and teeth and may negatively affect the equilibrium between demineralization and remineralization at the surface of the tooth. There is a need for alternatives to currently available commercial toothpastes that will remove dental calculus while maintaining the tooth's underlying structure. Calculus contains organic material (around 15%-20%), such as proteins, glycoproteins, lipids, DNA, carbohydrates, and bacteria as well as microparticles (formerly known as microfossils) like phytoliths and starch granules. Dental calculus is mostly inorganic, being composed mainly of calcium and phosphorus, with minor percentages of carbonate, sodium, magnesium, silicon, iron, and fluoride, in the form of minerals such as brushite, whitlockite, octacalcium phosphate, and hydroxyapatite. The calcium carbonate, on the other hand, might occur as two distinct minerals: calcite (CaCO₃) is the stable form, whereas aragonite is metastable and can eventually change into calcite with time or heat. Calcite is a mineral that has the same chemical composition as aragonite but a slightly different crystal structure. Aragonite lacks the rhombohedral cleavage of calcite and typically has needlelike crystals in its crystal form. Brushite and whitlockite are known to react favorably with the aragonite and calcite minerals, according to the following equations.

Dicalcium phosphate (Brushite) reaction with calcium carbonate:

3CaHPO₄·2H₂O+2CaCO₃→Ca₅(PO₄)₃OH+2CO₂+7H₂O (Eq. 1)

Tricalcium phosphate (whitlockite) reaction with calcium carbonate:

3Ca₃(PO₄)₂+H₂O+CaCO₃→2Ca₅(PO₄)₃OH+CO₂ (Eq.2)

When compared to the rhombohedral calcite crystals, the needle-like aragonite crystals may react faster. Hence the removal of the dental calculus could be facilitated by the reaction of the aragonite with brushite and whitlockite. The effectiveness of the aragonite and particularly a treated aragonite, as an appropriate treatment for calculus removal, compared to calcite (CaCO₃) found in conventional toothpastes was investigated.

The complex composition of toothpastes implies that it is necessary to ensure that the active ingredients are not inactivated in the process of production or delivery. For instance, calcium carbonate added to dentifrice binds to fluoride, rendering the latter ineffective as an anti-caries agent, which is highly undesirable (Shen et al. “Bioavailable fluoride in calcium-containing dentifrices” Scientific Reports, (2021) 11:146). Therefore, the composition of toothpastes is critical for their effectiveness on oral health maintenance and safety for the oral cavity.

In embodiments there is disclosed an oral care composition. The compositions of the present invention are oral care compositions containing as an ingredient treated aragonite calcium carbonate (CaCO₃) particles having more than 95% (w/w) calcium carbonate content, a specific surface area (SSA) of about 2.70 to about 3.1 m²/g, for use as a first dental abrasive. The compositions of the present invention also contain a fluoride compound suitable to provide beneficial fluoride treatment to teeth. Importantly, the treated aragonite calcium carbonate (CaCO₃) particles used in the present invention have been effectively treated in mildly acidic condition to avoid reaction of fluoride from the fluoride compound and the treated aragonite calcium carbonate particles.

Aragonite is a carbonate mineral, one of the two common, naturally occurring, crystal forms of calcium carbonate, CaCO₃, the other form being the mineral calcite. It is formed by biological and physical processes, including precipitation from marine and freshwater environments.

Aragonite's crystal lattice differs from that of calcite, resulting in a different crystal shape, an orthorhombic system with acicular crystals. Repeated twinning results in pseudo-hexagonal forms. Aragonite may be columnar or fibrous, occasionally in branching stalactitic forms called flos-ferri (“flowers of iron”) from their association with the ores at the Carinthian iron mines.

Aragonite forms naturally in almost all mollusk shells, and as the calcareous endoskeleton of warm- and cold-water corals (Scleractinia). Several serpulids have aragonitic tubes. Because the mineral deposition in mollusk shells is strongly biologically controlled, some crystal forms are distinctively different from those of inorganic aragonite. In some mollusks, the entire shell is aragonite; in others, aragonite forms only discrete parts of a bimineralic shell (aragonite plus calcite). Aragonite also forms in the ocean and in caves as inorganic precipitates called marine cements and speleothems, respectively. The nacreous layer of the aragonite fossil shells of some extinct ammonites forms an iridescent material called ammolite. Ammolite is primarily aragonite with impurities that make it iridescent and valuable as a gemstone.

According to embodiments, the aragonite calcium carbonate (CaCO₃) may be from any suitable origin that is capable of providing the treated aragonite calcium carbonate (CaCO₃) particles. In embodiments, the aragonite calcium carbonate may be from animal origin, for example from cuttlefish or shellfish origin. According to another embodiment, the aragonite calcium carbonate may be from vegetal origin, for example from oolitic origin. In a preferred embodiment, the aragonite calcium carbonate and the treated aragonite calcium carbonate are free of chitin, to avoid allergic reactions to those individuals that are allergic to it. As chitin is normally present in aragonites sourced from animal origin, aragonites from vegetal origin, for example from oolitic origin are preferred when wanting to avoid the presence of chitin.

The particles of treated aragonite calcium carbonate (CaCO₃) used in the present invention may be comprised particles of the treated aragonite calcium carbonate particles which have a particle size of from about 25 μm to about 70 μm, or from about 26 μm to about 70 μm, or from about 27 μm to about 70 μm, or from about 28 μm to about 70 μm, or from about 29 μm to about 70 μm, or from about 30 μm to about 70 μm, or from about 31 μm to about 70 μm, or from about 32 μm to about 70 μm, or from about 33 μm to about 70 μm, or from about 34 μm to about 70 μm, or from about 35 μm to about 70 μm, or from about 36 μm to about 70 μm, or from about 37 μm to about 70 μm, or from about 38 μm to about 70 μm, or from about 39 μm to about 70 μm, or from about 40 μm to about 70 μm, or from about 41 μm to about 70 μm, or from about 42 μm to about 70 μm, or from about 43 μm to about 70 μm, or from about 44 μm to about 70 μm, or from about 45 μm to about 70 μm, or from about 46 μm to about 70 μm, or from about 47 μm to about 70 μm, or from about 48 μm to about 70 μm, or from about 49 μm to about 70 μm, or from about 50 μm to about 70 μm, or from about 51 μm to about 70 μm, or from about 52 μm to about 70 μm, or from about 53 μm to about 70 μm, or from about 54 μm to about 70 μm, or from about 55 μm to about 70 μm, or from about 56 μm to about 70 μm, or from about 57 μm to about 70 μm, or from about 58 μm to about 70 μm, or from about 59 μm to about 70 μm, or from about 60 μm to about 70 μm, or from about 61 μm to about 70 μm, or from about 62 μm to about 70 μm, or from about 63 μm to about 70 μm, or from about 64 μm to about 70 μm, or from about 65 μm to about 70 μm, or from about 66 μm to about 70 μm, or from about 67 μm to about 70 μm, or from about 68 μm to about 70 μm, or from about 69 μm to about 70 μm, or from about 25 μm to about 69 μm, or from about 26 μm to about 69 μm, or from about 27 μm to about 69 μm, or from about 28 μm to about 69 μm, or from about 29 μm to about 69 μm, or from about 30 μm to about 69 μm, or from about 31 μm to about 69 μm, or from about 32 μm to about 69 μm, or from about 33 μm to about 69 μm, or from about 34 μm to about 69 μm, or from about 35 μm to about 69 μm, or from about 36 μm to about 69 μm, or from about 37 μm to about 69 μm, or from about 38 μm to about 69 μm, or from about 39 μm to about 69 μm, or from about 40 μm to about 69 μm, or from about 41 μm to about 69 μm, or from about 42 μm to about 69 μm, or from about 43 μm to about 69 μm, or from about 44 μm to about 69 μm, or from about 45 μm to about 69 μm, or from about 46 μm to about 69 μm, or from about 47 μm to about 69 μm, or from about 48 μm to about 69 μm, or from about 49 μm to about 69 μm, or from about 50 μm to about 69 μm, or from about 51 μm to about 69 μm, or from about 52 μm to about 69 μm, or from about 53 μm to about 69 μm, or from about 54 μm to about 69 μm, or from about 55 μm to about 69 μm, or from about 56 μm to about 69 μm, or from about 57 μm to about 69 μm, or from about 58 μm to about 69 μm, or from about 59 μm to about 69 μm, or from about 60 μm to about 69 μm, or from about 61 μm to about 69 μm, or from about 62 μm to about 69 μm, or from about 63 μm to about 69 μm, or from about 64 μm to about 69 μm, or from about 65 μm to about 69 μm, or from about 66 μm to about 69 μm, or from about 67 μm to about 69 μm, or from about 68 μm to about 69 μm, or from about 25 μm to about 68 μm, or from about 26 μm to about 68 μm, or from about 27 μm to about 68 μm, or from about 28 μm to about 68 μm, or from about 29 μm to about 68 μm, or from about 30 μm to about 68 μm, or from about 31 μm to about 68 μm, or from about 32 μm to about 68 μm, or from about 33 μm to about 68 μm, or from about 34 μm to about 68 μm, or from about 35 μm to about 68 μm, or from about 36 μm to about 68 μm, or from about 37 μm to about 68 μm, or from about 38 μm to about 68 μm, or from about 39 μm to about 68 μm, or from about 40 μm to about 68 μm, or from about 41 μm to about 68 μm, or from about 42 μm to about 68 μm, or from about 43 μm to about 68 μm, or from about 44 μm to about 68 μm, or from about 45 μm to about 68 μm, or from about 46 μm to about 68 μm, or from about 47 μm to about 68 μm, or from about 48 μm to about 68 μm, or from about 49 μm to about 68 μm, or from about 50 μm to about 68 μm, or from about 51 μm to about 68 μm, or from about 52 μm to about 68 μm, or from about 53 μm to about 68 μm, or from about 54 μm to about 68 μm, or from about 55 μm to about 68 μm, or from about 56 μm to about 68 μm, or from about 57 μm to about 68 μm, or from about 58 μm to about 68 μm, or from about 59 μm to about 68 μm, or from about 60 μm to about 68 μm, or from about 61 μm to about 68 μm, or from about 62 μm to about 68 μm, or from about 63 μm to about 68 μm, or from about 64 μm to about 68 μm, or from about 65 μm to about 68 μm, or from about 66 μm to about 68 μm, or from about 67 μm to about 68 μm, or from about 25 μm to about 67 μm, or from about 26 μm to about 67 μm, or from about 27 μm to about 67 μm, or from about 28 μm to about 67 μm, or from about 29 μm to about 67 μm, or from about 30 μm to about 67 μm, or from about 31 μm to about 67 μm, or from about 32 μm to about 67 μm, or from about 33 μm to about 67 μm, or from about 34 μm to about 67 μm, or from about 35 μm to about 67 μm, or from about 36 μm to about 67 μm, or from about 37 μm to about 67 μm, or from about 38 μm to about 67 μm, or from about 39 μm to about 67 μm, or from about 40 μm to about 67 μm, or from about 41 μm to about 67 μm, or from about 42 μm to about 67 μm, or from about 43 μm to about 67 μm, or from about 44 μm to about 67 μm, or from about 45 μm to about 67 μm, or from about 46 μm to about 67 μm, or from about 47 μm to about 67 μm, or from about 48 μm to about 67 μm, or from about 49 μm to about 67 μm, or from about 50 μm to about 67 μm, or from about 51 μm to about 67 μm, or from about 52 μm to about 67 μm, or from about 53 μm to about 67 μm, or from about 54 μm to about 67 μm, or from about 55 μm to about 67 μm, or from about 56 μm to about 67 μm, or from about 57 μm to about 67 μm, or from about 58 μm to about 67 μm, or from about 59 μm to about 67 μm, or from about 60 μm to about 67 μm, or from about 61 μm to about 67 μm, or from about 62 μm to about 67 μm, or from about 63 μm to about 67 μm, or from about 64 μm to about 67 μm, or from about 65 μm to about 67 μm, or from about 66 μm to about 67 μm, or from about 25 μm to about 66 μm, or from about 26 μm to about 66 μm, or from about 27 μm to about 66 μm, or from about 28 μm to about 66 μm, or from about 29 μm to about 66 μm, or from about 30 μm to about 66 μm, or from about 31 μm to about 66 μm, or from about 32 μm to about 66 μm, or from about 33 μm to about 66 μm, or from about 34 μm to about 66 μm, or from about 35 μm to about 66 μm, or from about 36 μm to about 66 μm, or from about 37 μm to about 66 μm, or from about 38 μm to about 66 μm, or from about 39 μm to about 66 μm, or from about 40 μm to about 66 μm, or from about 41 μm to about 66 μm, or from about 42 μm to about 66 μm, or from about 43 μm to about 66 μm, or from about 44 μm to about 66 μm, or from about 45 μm to about 66 μm, or from about 46 μm to about 66 μm, or from about 47 μm to about 66 μm, or from about 48 μm to about 66 μm, or from about 49 μm to about 66 μm, or from about 50 μm to about 66 μm, or from about 51 μm to about 66 μm, or from about 52 μm to about 66 μm, or from about 53 μm to about 66 μm, or from about 54 μm to about 66 μm, or from about 55 μm to about 66 μm, or from about 56 μm to about 66 μm, or from about 57 μm to about 66 μm, or from about 58 μm to about 66 μm, or from about 59 μm to about 66 μm, or from about 60 μm to about 66 μm, or from about 61 μm to about 66 μm, or from about 62 μm to about 66 μm, or from about 63 μm to about 66 μm, or from about 64 μm to about 66 μm, or from about 65 μm to about 66 μm, or from about 25 μm to about 65 μm, or from about 26 μm to about 65 μm, or from about 27 μm to about 65 μm, or from about 28 μm to about 65 μm, or from about 29 μm to about 65 μm, or from about 30 μm to about 65 μm, or from about 31 μm to about 65 μm, or from about 32 μm to about 65 μm, or from about 33 μm to about 65 μm, or from about 34 μm to about 65 μm, or from about 35 μm to about 65 μm, or from about 36 μm to about 65 μm, or from about 37 μm to about 65 μm, or from about 38 μm to about 65 μm, or from about 39 μm to about 65 μm, or from about 40 μm to about 65 μm, or from about 41 μm to about 65 μm, or from about 42 μm to about 65 μm, or from about 43 μm to about 65 μm, or from about 44 μm to about 65 μm, or from about 45 μm to about 65 μm, or from about 46 μm to about 65 μm, or from about 47 μm to about 65 μm, or from about 48 μm to about 65 μm, or from about 49 μm to about 65 μm, or from about 50 μm to about 65 μm, or from about 51 μm to about 65 μm, or from about 52 μm to about 65 μm, or from about 53 μm to about 65 μm, or from about 54 μm to about 65 μm, or from about 55 μm to about 65 μm, or from about 56 μm to about 65 μm, or from about 57 μm to about 65 μm, or from about 58 μm to about 65 μm, or from about 59 μm to about 65 μm, or from about 60 μm to about 65 μm, or from about 61 μm to about 65 μm, or from about 62 μm to about 65 μm, or from about 63 μm to about 65 μm, or from about 64 μm to about 65 μm, or from about 25 μm to about 64 μm, or from about 26 μm to about 64 μm, or from about 27 μm to about 64 μm, or from about 28 μm to about 64 μm, or from about 29 μm to about 64 μm, or from about 30 μm to about 64 μm, or from about 31 μm to about 64 μm, or from about 32 μm to about 64 μm, or from about 33 μm to about 64 μm, or from about 34 μm to about 64 μm, or from about 35 μm to about 64 μm, or from about 36 μm to about 64 μm, or from about 37 μm to about 64 μm, or from about 38 μm to about 64 μm, or from about 39 μm to about 64 μm, or from about 40 μm to about 64 μm, or from about 41 μm to about 64 μm, or from about 42 μm to about 64 μm, or from about 43 μm to about 64 μm, or from about 44 μm to about 64 μm, or from about 45 μm to about 64 μm, or from about 46 μm to about 64 μm, or from about 47 μm to about 64 μm, or from about 48 μm to about 64 μm, or from about 49 μm to about 64 μm, or from about 50 μm to about 64 μm, or from about 51 μm to about 64 μm, or from about 52 μm to about 64 μm, or from about 53 μm to about 64 μm, or from about 54 μm to about 64 μm, or from about 55 μm to about 64 μm, or from about 56 μm to about 64 μm, or from about 57 μm to about 64 μm, or from about 58 μm to about 64 μm, or from about 59 μm to about 64 μm, or from about 60 μm to about 64 μm, or from about 61 μm to about 64 μm, or from about 62 μm to about 64 μm, or from about 63 μm to about 64 μm, or from about 25 μm to about 63 μm, or from about 26 μm to about 63 μm, or from about 27 μm to about 63 μm, or from about 28 μm to about 63 μm, or from about 29 μm to about 63 μm, or from about 30 μm to about 63 μm, or from about 31 μm to about 63 μm, or from about 32 μm to about 63 μm, or from about 33 μm to about 63 μm, or from about 34 μm to about 63 μm, or from about 35 μm to about 63 μm, or from about 36 μm to about 63 μm, or from about 37 μm to about 63 μm, or from about 38 μm to about 63 μm, or from about 39 μm to about 63 μm, or from about 40 μm to about 63 μm, or from about 41 μm to about 63 μm, or from about 42 μm to about 63 μm, or from about 43 μm to about 63 μm, or from about 44 μm to about 63 μm, or from about 45 μm to about 63 μm, or from about 46 μm to about 63 μm, or from about 47 μm to about 63 μm, or from about 48 μm to about 63 μm, or from about 49 μm to about 63 μm, or from about 50 μm to about 63 μm, or from about 51 μm to about 63 μm, or from about 52 μm to about 63 μm, or from about 53 μm to about 63 μm, or from about 54 μm to about 63 μm, or from about 55 μm to about 63 μm, or from about 56 μm to about 63 μm, or from about 57 μm to about 63 μm, or from about 58 μm to about 63 μm, or from about 59 μm to about 63 μm, or from about 60 μm to about 63 μm, or from about 61 μm to about 63 μm, or from about 62 μm to about 63 μm, or from about 25 μm to about 62 μm, or from about 26 μm to about 62 μm, or from about 27 μm to about 62 μm, or from about 28 μm to about 62 μm, or from about 29 μm to about 62 μm, or from about 30 μm to about 62 μm, or from about 31 μm to about 62 μm, or from about 32 μm to about 62 μm, or from about 33 μm to about 62 μm, or from about 34 μm to about 62 μm, or from about 35 μm to about 62 μm, or from about 36 μm to about 62 μm, or from about 37 μm to about 62 μm, or from about 38 μm to about 62 μm, or from about 39 μm to about 62 μm, or from about 40 μm to about 62 μm, or from about 41 μm to about 62 μm, or from about 42 μm to about 62 μm, or from about 43 μm to about 62 μm, or from about 44 μm to about 62 μm, or from about 45 μm to about 62 μm, or from about 46 μm to about 62 μm, or from about 47 μm to about 62 μm, or from about 48 μm to about 62 μm, or from about 49 μm to about 62 μm, or from about 50 μm to about 62 μm, or from about 51 μm to about 62 μm, or from about 52 μm to about 62 μm, or from about 53 μm to about 62 μm, or from about 54 μm to about 62 μm, or from about 55 μm to about 62 μm, or from about 56 μm to about 62 μm, or from about 57 μm to about 62 μm, or from about 58 μm to about 62 μm, or from about 59 μm to about 62 μm, or from about 60 μm to about 62 μm, or from about 61 μm to about 62 μm, or from about 25 μm to about 61 μm, or from about 26 μm to about 61 μm, or from about 27 μm to about 61 μm, or from about 28 μm to about 61 μm, or from about 29 μm to about 61 μm, or from about 30 μm to about 61 μm, or from about 31 μm to about 61 μm, or from about 32 μm to about 61 μm, or from about 33 μm to about 61 μm, or from about 34 μm to about 61 μm, or from about 35 μm to about 61 μm, or from about 36 μm to about 61 μm, or from about 37 μm to about 61 μm, or from about 38 μm to about 61 μm, or from about 39 μm to about 61 μm, or from about 40 μm to about 61 μm, or from about 41 μm to about 61 μm, or from about 42 μm to about 61 μm, or from about 43 μm to about 61 μm, or from about 44 μm to about 61 μm, or from about 45 μm to about 61 μm, or from about 46 μm to about 61 μm, or from about 47 μm to about 61 μm, or from about 48 μm to about 61 μm, or from about 49 μm to about 61 μm, or from about 50 μm to about 61 μm, or from about 51 μm to about 61 μm, or from about 52 μm to about 61 μm, or from about 53 μm to about 61 μm, or from about 54 μm to about 61 μm, or from about 55 μm to about 61 μm, or from about 56 μm to about 61 μm, or from about 57 μm to about 61 μm, or from about 58 μm to about 61 μm, or from about 59 μm to about 61 μm, or from about 60 μm to about 61 μm, or from about 25 μm to about 60 μm, or from about 26 μm to about 60 μm, or from about 27 μm to about 60 μm, or from about 28 μm to about 60 μm, or from about 29 μm to about 60 μm, or from about 30 μm to about 60 μm, or from about 31 μm to about 60 μm, or from about 32 μm to about 60 μm, or from about 33 μm to about 60 μm, or from about 34 μm to about 60 μm, or from about 35 μm to about 60 μm, or from about 36 μm to about 60 μm, or from about 37 μm to about 60 μm, or from about 38 μm to about 60 μm, or from about 39 μm to about 60 μm, or from about 40 μm to about 60 μm, or from about 41 μm to about 60 μm, or from about 42 μm to about 60 μm, or from about 43 μm to about 60 μm, or from about 44 μm to about 60 μm, or from about 45 μm to about 60 μm, or from about 46 μm to about 60 μm, or from about 47 μm to about 60 μm, or from about 48 μm to about 60 μm, or from about 49 μm to about 60 μm, or from about 50 μm to about 60 μm, or from about 51 μm to about 60 μm, or from about 52 μm to about 60 μm, or from about 53 μm to about 60 μm, or from about 54 μm to about 60 μm, or from about 55 μm to about 60 μm, or from about 56 μm to about 60 μm, or from about 57 μm to about 60 μm, or from about 58 μm to about 60 μm, or from about 59 μm to about 60 μm, or from about 25 μm to about 59 μm, or from about 26 μm to about 59 μm, or from about 27 μm to about 59 μm, or from about 28 μm to about 59 μm, or from about 29 μm to about 59 μm, or from about 30 μm to about 59 μm, or from about 31 μm to about 59 μm, or from about 32 μm to about 59 μm, or from about 33 μm to about 59 μm, or from about 34 μm to about 59 μm, or from about 35 μm to about 59 μm, or from about 36 μm to about 59 μm, or from about 37 μm to about 59 μm, or from about 38 μm to about 59 μm, or from about 39 μm to about 59 μm, or from about 40 μm to about 59 μm, or from about 41 μm to about 59 μm, or from about 42 μm to about 59 μm, or from about 43 μm to about 59 μm, or from about 44 μm to about 59 μm, or from about 45 μm to about 59 μm, or from about 46 μm to about 59 μm, or from about 47 μm to about 59 μm, or from about 48 μm to about 59 μm, or from about 49 μm to about 59 μm, or from about 50 μm to about 59 μm, or from about 51 μm to about 59 μm, or from about 52 μm to about 59 μm, or from about 53 μm to about 59 μm, or from about 54 μm to about 59 μm, or from about 55 μm to about 59 μm, or from about 56 μm to about 59 μm, or from about 57 μm to about 59 μm, or from about 58 μm to about 59 μm, or from about 25 μm to about 58 μm, or from about 26 μm to about 58 μm, or from about 27 μm to about 58 μm, or from about 28 μm to about 58 μm, or from about 29 μm to about 58 μm, or from about 30 μm to about 58 μm, or from about 31 μm to about 58 μm, or from about 32 μm to about 58 μm, or from about 33 μm to about 58 μm, or from about 34 μm to about 58 μm, or from about 35 μm to about 58 μm, or from about 36 μm to about 58 μm, or from about 37 μm to about 58 μm, or from about 38 μm to about 58 μm, or from about 39 μm to about 58 μm, or from about 40 μm to about 58 μm, or from about 41 μm to about 58 μm, or from about 42 μm to about 58 μm, or from about 43 μm to about 58 μm, or from about 44 μm to about 58 μm, or from about 45 μm to about 58 μm, or from about 46 μm to about 58 μm, or from about 47 μm to about 58 μm, or from about 48 μm to about 58 μm, or from about 49 μm to about 58 μm, or from about 50 μm to about 58 μm, or from about 51 μm to about 58 μm, or from about 52 μm to about 58 μm, or from about 53 μm to about 58 μm, or from about 54 μm to about 58 μm, or from about 55 μm to about 58 μm, or from about 56 μm to about 58 μm, or from about 57 μm to about 58 μm, or from about 25 μm to about 57 μm, or from about 26 μm to about 57 μm, or from about 27 μm to about 57 μm, or from about 28 μm to about 57 μm, or from about 29 μm to about 57 μm, or from about 30 μm to about 57 μm, or from about 31 μm to about 57 μm, or from about 32 μm to about 57 μm, or from about 33 μm to about 57 μm, or from about 34 μm to about 57 μm, or from about 35 μm to about 57 μm, or from about 36 μm to about 57 μm, or from about 37 μm to about 57 μm, or from about 38 μm to about 57 μm, or from about 39 μm to about 57 μm, or from about 40 μm to about 57 μm, or from about 41 μm to about 57 μm, or from about 42 μm to about 57 μm, or from about 43 μm to about 57 μm, or from about 44 μm to about 57 μm, or from about 45 μm to about 57 μm, or from about 46 μm to about 57 μm, or from about 47 μm to about 57 μm, or from about 48 μm to about 57 μm, or from about 49 μm to about 57 μm, or from about 50 μm to about 57 μm, or from about 51 μm to about 57 μm, or from about 52 μm to about 57 μm, or from about 53 μm to about 57 μm, or from about 54 μm to about 57 μm, or from about 55 μm to about 57 μm, or from about 56 μm to about 57 μm, or from about 25 μm to about 56 μm, or from about 26 μm to about 56 μm, or from about 27 μm to about 56 μm, or from about 28 μm to about 56 μm, or from about 29 μm to about 56 μm, or from about 30 μm to about 56 μm, or from about 31 μm to about 56 μm, or from about 32 μm to about 56 μm, or from about 33 μm to about 56 μm, or from about 34 μm to about 56 μm, or from about 35 μm to about 56 μm, or from about 36 μm to about 56 μm, or from about 37 μm to about 56 μm, or from about 38 μm to about 56 μm, or from about 39 μm to about 56 μm, or from about 40 μm to about 56 μm, or from about 41 μm to about 56 μm, or from about 42 μm to about 56 μm, or from about 43 μm to about 56 μm, or from about 44 μm to about 56 μm, or from about 45 μm to about 56 μm, or from about 46 μm to about 56 μm, or from about 47 μm to about 56 μm, or from about 48 μm to about 56 μm, or from about 49 μm to about 56 μm, or from about 50 μm to about 56 μm, or from about 51 μm to about 56 μm, or from about 52 μm to about 56 μm, or from about 53 μm to about 56 μm, or from about 54 μm to about 56 μm, or from about 55 μm to about 56 μm, or from about 25 μm to about 55 μm, or from about 26 μm to about 55 μm, or from about 27 μm to about 55 μm, or from about 28 μm to about 55 μm, or from about 29 μm to about 55 μm, or from about 30 μm to about 55 μm, or from about 31 μm to about 55 μm, or from about 32 μm to about 55 μm, or from about 33 μm to about 55 μm, or from about 34 μm to about 55 μm, or from about 35 μm to about 55 μm, or from about 36 μm to about 55 μm, or from about 37 μm to about 55 μm, or from about 38 μm to about 55 μm, or from about 39 μm to about 55 μm, or from about 40 μm to about 55 μm, or from about 41 μm to about 55 μm, or from about 42 μm to about 55 μm, or from about 43 μm to about 55 μm, or from about 44 μm to about 55 μm, or from about 45 μm to about 55 μm, or from about 46 μm to about 55 μm, or from about 47 μm to about 55 μm, or from about 48 μm to about 55 μm, or from about 49 μm to about 55 μm, or from about 50 μm to about 55 μm, or from about 51 μm to about 55 μm, or from about 52 μm to about 55 μm, or from about 53 μm to about 55 μm, or from about 54 μm to about 55 μm, or from about 25 μm to about 54 μm, or from about 26 μm to about 54 μm, or from about 27 μm to about 54 μm, or from about 28 μm to about 54 μm, or from about 29 μm to about 54 μm, or from about 30 μm to about 54 μm, or from about 31 μm to about 54 μm, or from about 32 μm to about 54 μm, or from about 33 μm to about 54 μm, or from about 34 μm to about 54 μm, or from about 35 μm to about 54 μm, or from about 36 μm to about 54 μm, or from about 37 μm to about 54 μm, or from about 38 μm to about 54 μm, or from about 39 μm to about 54 μm, or from about 40 μm to about 54 μm, or from about 41 μm to about 54 μm, or from about 42 μm to about 54 μm, or from about 43 μm to about 54 μm, or from about 44 μm to about 54 μm, or from about 45 μm to about 54 μm, or from about 46 μm to about 54 μm, or from about 47 μm to about 54 μm, or from about 48 μm to about 54 μm, or from about 49 μm to about 54 μm, or from about 50 μm to about 54 μm, or from about 51 μm to about 54 μm, or from about 52 μm to about 54 μm, or from about 53 μm to about 54 μm, or from about 25 μm to about 53 μm, or from about 26 μm to about 53 μm, or from about 27 μm to about 53 μm, or from about 28 μm to about 53 μm, or from about 29 μm to about 53 μm, or from about 30 μm to about 53 μm, or from about 31 μm to about 53 μm, or from about 32 μm to about 53 μm, or from about 33 μm to about 53 μm, or from about 34 μm to about 53 μm, or from about 35 μm to about 53 μm, or from about 36 μm to about 53 μm, or from about 37 μm to about 53 μm, or from about 38 μm to about 53 μm, or from about 39 μm to about 53 μm, or from about 40 μm to about 53 μm, or from about 41 μm to about 53 μm, or from about 42 μm to about 53 μm, or from about 43 μm to about 53 μm, or from about 44 μm to about 53 μm, or from about 45 μm to about 53 μm, or from about 46 μm to about 53 μm, or from about 47 μm to about 53 μm, or from about 48 μm to about 53 μm, or from about 49 μm to about 53 μm, or from about 50 μm to about 53 μm, or from about 51 μm to about 53 μm, or from about 52 μm to about 53 μm, or from about 25 μm to about 52 μm, or from about 26 μm to about 52 μm, or from about 27 μm to about 52 μm, or from about 28 μm to about 52 μm, or from about 29 μm to about 52 μm, or from about 30 μm to about 52 μm, or from about 31 μm to about 52 μm, or from about 32 μm to about 52 μm, or from about 33 μm to about 52 μm, or from about 34 μm to about 52 μm, or from about 35 μm to about 52 μm, or from about 36 μm to about 52 μm, or from about 37 μm to about 52 μm, or from about 38 μm to about 52 μm, or from about 39 μm to about 52 μm, or from about 40 μm to about 52 μm, or from about 41 μm to about 52 μm, or from about 42 μm to about 52 μm, or from about 43 μm to about 52 μm, or from about 44 μm to about 52 μm, or from about 45 μm to about 52 μm, or from about 46 μm to about 52 μm, or from about 47 μm to about 52 μm, or from about 48 μm to about 52 μm, or from about 49 μm to about 52 μm, or from about 50 μm to about 52 μm, or from about 51 μm to about 52 μm, or from about 25 μm to about 51 μm, or from about 26 μm to about 51 μm, or from about 27 μm to about 51 μm, or from about 28 μm to about 51 μm, or from about 29 μm to about 51 μm, or from about 30 μm to about 51 μm, or from about 31 μm to about 51 μm, or from about 32 μm to about 51 μm, or from about 33 μm to about 51 μm, or from about 34 μm to about 51 μm, or from about 35 μm to about 51 μm, or from about 36 μm to about 51 μm, or from about 37 μm to about 51 μm, or from about 38 μm to about 51 μm, or from about 39 μm to about 51 μm, or from about 40 μm to about 51 μm, or from about 41 μm to about 51 μm, or from about 42 μm to about 51 μm, or from about 43 μm to about 51 μm, or from about 44 μm to about 51 μm, or from about 45 μm to about 51 μm, or from about 46 μm to about 51 μm, or from about 47 μm to about 51 μm, or from about 48 μm to about 51 μm, or from about 49 μm to about 51 μm, or from about 50 μm to about 51 μm, or from about 25 μm to about 50 μm, or from about 26 μm to about 50 μm, or from about 27 μm to about 50 μm, or from about 28 μm to about 50 μm, or from about 29 μm to about 50 μm, or from about 30 μm to about 50 μm, or from about 31 μm to about 50 μm, or from about 32 μm to about 50 μm, or from about 33 μm to about 50 μm, or from about 34 μm to about 50 μm, or from about 35 μm to about 50 μm, or from about 36 μm to about 50 μm, or from about 37 μm to about 50 μm, or from about 38 μm to about 50 μm, or from about 39 μm to about 50 μm, or from about 40 μm to about 50 μm, or from about 41 μm to about 50 μm, or from about 42 μm to about 50 μm, or from about 43 μm to about 50 μm, or from about 44 μm to about 50 μm, or from about 45 μm to about 50 μm, or from about 46 μm to about 50 μm, or from about 47 μm to about 50 μm, or from about 48 μm to about 50 μm, or from about 49 μm to about 50 μm, or from about 25 μm to about 49 μm, or from about 26 μm to about 49 μm, or from about 27 μm to about 49 μm, or from about 28 μm to about 49 μm, or from about 29 μm to about 49 μm, or from about 30 μm to about 49 μm, or from about 31 μm to about 49 μm, or from about 32 μm to about 49 μm, or from about 33 μm to about 49 μm, or from about 34 μm to about 49 μm, or from about 35 μm to about 49 μm, or from about 36 μm to about 49 μm, or from about 37 μm to about 49 μm, or from about 38 μm to about 49 μm, or from about 39 μm to about 49 μm, or from about 40 μm to about 49 μm, or from about 41 μm to about 49 μm, or from about 42 μm to about 49 μm, or from about 43 μm to about 49 μm, or from about 44 μm to about 49 μm, or from about 45 μm to about 49 μm, or from about 46 μm to about 49 μm, or from about 47 μm to about 49 μm, or from about 48 μm to about 49 μm, or from about 25 μm to about 48 μm, or from about 26 μm to about 48 μm, or from about 27 μm to about 48 μm, or from about 28 μm to about 48 μm, or from about 29 μm to about 48 μm, or from about 30 μm to about 48 μm, or from about 31 μm to about 48 μm, or from about 32 μm to about 48 μm, or from about 33 μm to about 48 μm, or from about 34 μm to about 48 μm, or from about 35 μm to about 48 μm, or from about 36 μm to about 48 μm, or from about 37 μm to about 48 μm, or from about 38 μm to about 48 μm, or from about 39 μm to about 48 μm, or from about 40 μm to about 48 μm, or from about 41 μm to about 48 μm, or from about 42 μm to about 48 μm, or from about 43 μm to about 48 μm, or from about 44 μm to about 48 μm, or from about 45 μm to about 48 μm, or from about 46 μm to about 48 μm, or from about 47 μm to about 48 μm, or from about 25 μm to about 47 μm, or from about 26 μm to about 47 μm, or from about 27 μm to about 47 μm, or from about 28 μm to about 47 μm, or from about 29 μm to about 47 μm, or from about 30 μm to about 47 μm, or from about 31 μm to about 47 μm, or from about 32 μm to about 47 μm, or from about 33 μm to about 47 μm, or from about 34 μm to about 47 μm, or from about 35 μm to about 47 μm, or from about 36 μm to about 47 μm, or from about 37 μm to about 47 μm, or from about 38 μm to about 47 μm, or from about 39 μm to about 47 μm, or from about 40 μm to about 47 μm, or from about 41 μm to about 47 μm, or from about 42 μm to about 47 μm, or from about 43 μm to about 47 μm, or from about 44 μm to about 47 μm, or from about 45 μm to about 47 μm, or from about 46 μm to about 47 μm, or from about 25 μm to about 46 μm, or from about 26 μm to about 46 μm, or from about 27 μm to about 46 μm, or from about 28 μm to about 46 μm, or from about 29 μm to about 46 μm, or from about 30 μm to about 46 μm, or from about 31 μm to about 46 μm, or from about 32 μm to about 46 μm, or from about 33 μm to about 46 μm, or from about 34 μm to about 46 μm, or from about 35 μm to about 46 μm, or from about 36 μm to about 46 μm, or from about 37 μm to about 46 μm, or from about 38 μm to about 46 μm, or from about 39 μm to about 46 μm, or from about 40 μm to about 46 μm, or from about 41 μm to about 46 μm, or from about 42 μm to about 46 μm, or from about 43 μm to about 46 μm, or from about 44 μm to about 46 μm, or from about 45 μm to about 46 μm, or from about 25 μm to about 45 μm, or from about 26 μm to about 45 μm, or from about 27 μm to about 45 μm, or from about 28 μm to about 45 μm, or from about 29 μm to about 45 μm, or from about 30 μm to about 45 μm, or from about 31 μm to about 45 μm, or from about 32 μm to about 45 μm, or from about 33 μm to about 45 μm, or from about 34 μm to about 45 μm, or from about 35 μm to about 45 μm, or from about 36 μm to about 45 μm, or from about 37 μm to about 45 μm, or from about 38 μm to about 45 μm, or from about 39 μm to about 45 μm, or from about 40 μm to about 45 μm, or from about 41 μm to about 45 μm, or from about 42 μm to about 45 μm, or from about 43 μm to about 45 μm, or from about 44 μm to about 45 μm, or from about 25 μm to about 44 μm, or from about 26 μm to about 44 μm, or from about 27 μm to about 44 μm, or from about 28 μm to about 44 μm, or from about 29 μm to about 44 μm, or from about 30 μm to about 44 μm, or from about 31 μm to about 44 μm, or from about 32 μm to about 44 μm, or from about 33 μm to about 44 μm, or from about 34 μm to about 44 μm, or from about 35 μm to about 44 μm, or from about 36 μm to about 44 μm, or from about 37 μm to about 44 μm, or from about 38 μm to about 44 μm, or from about 39 μm to about 44 μm, or from about 40 μm to about 44 μm, or from about 41 μm to about 44 μm, or from about 42 μm to about 44 μm, or from about 43 μm to about 44 μm, or from about 25 μm to about 43 μm, or from about 26 μm to about 43 μm, or from about 27 μm to about 43 μm, or from about 28 μm to about 43 μm, or from about 29 μm to about 43 μm, or from about 30 μm to about 43 μm, or from about 31 μm to about 43 μm, or from about 32 μm to about 43 μm, or from about 33 μm to about 43 μm, or from about 34 μm to about 43 μm, or from about 35 μm to about 43 μm, or from about 36 μm to about 43 μm, or from about 37 μm to about 43 μm, or from about 38 μm to about 43 μm, or from about 39 μm to about 43 μm, or from about 40 μm to about 43 μm, or from about 41 μm to about 43 μm, or from about 42 μm to about 43 μm, or from about 25 μm to about 42 μm, or from about 26 μm to about 42 μm, or from about 27 μm to about 42 μm, or from about 28 μm to about 42 μm, or from about 29 μm to about 42 μm, or from about 30 μm to about 42 μm, or from about 31 μm to about 42 μm, or from about 32 μm to about 42 μm, or from about 33 μm to about 42 μm, or from about 34 μm to about 42 μm, or from about 35 μm to about 42 μm, or from about 36 μm to about 42 μm, or from about 37 μm to about 42 μm, or from about 38 μm to about 42 μm, or from about 39 μm to about 42 μm, or from about 40 μm to about 42 μm, or from about 41 μm to about 42 μm, or from about 25 μm to about 41 μm, or from about 26 μm to about 41 μm, or from about 27 μm to about 41 μm, or from about 28 μm to about 41 μm, or from about 29 μm to about 41 μm, or from about 30 μm to about 41 μm, or from about 31 μm to about 41 μm, or from about 32 μm to about 41 μm, or from about 33 μm to about 41 μm, or from about 34 μm to about 41 μm, or from about 35 μm to about 41 μm, or from about 36 μm to about 41 μm, or from about 37 μm to about 41 μm, or from about 38 μm to about 41 μm, or from about 39 μm to about 41 μm, or from about 40 μm to about 41 μm, or from about 25 μm to about 40 μm, or from about 26 μm to about 40 μm, or from about 27 μm to about 40 μm, or from about 28 μm to about 40 μm, or from about 29 μm to about 40 μm, or from about 30 μm to about 40 μm, or from about 31 μm to about 40 μm, or from about 32 μm to about 40 μm, or from about 33 μm to about 40 μm, or from about 34 μm to about 40 μm, or from about 35 μm to about 40 μm, or from about 36 μm to about 40 μm, or from about 37 μm to about 40 μm, or from about 38 μm to about 40 μm, or from about 39 μm to about 40 μm, or from about 25 μm to about 39 μm, or from about 26 μm to about 39 μm, or from about 27 μm to about 39 μm, or from about 28 μm to about 39 μm, or from about 29 μm to about 39 μm, or from about 30 μm to about 39 μm, or from about 31 μm to about 39 μm, or from about 32 μm to about 39 μm, or from about 33 μm to about 39 μm, or from about 34 μm to about 39 μm, or from about 35 μm to about 39 μm, or from about 36 μm to about 39 μm, or from about 37 μm to about 39 μm, or from about 38 μm to about 39 μm, or from about 25 μm to about 38 μm, or from about 26 μm to about 38 μm, or from about 27 μm to about 38 μm, or from about 28 μm to about 38 μm, or from about 29 μm to about 38 μm, or from about 30 μm to about 38 μm, or from about 31 μm to about 38 μm, or from about 32 μm to about 38 μm, or from about 33 μm to about 38 μm, or from about 34 μm to about 38 μm, or from about 35 μm to about 38 μm, or from about 36 μm to about 38 μm, or from about 37 μm to about 38 μm, or from about 25 μm to about 37 μm, or from about 26 μm to about 37 μm, or from about 27 μm to about 37 μm, or from about 28 μm to about 37 μm, or from about 29 μm to about 37 μm, or from about 30 μm to about 37 μm, or from about 31 μm to about 37 μm, or from about 32 μm to about 37 μm, or from about 33 μm to about 37 μm, or from about 34 μm to about 37 μm, or from about 35 μm to about 37 μm, or from about 36 μm to about 37 μm, or from about 25 μm to about 36 μm, or from about 26 μm to about 36 μm, or from about 27 μm to about 36 μm, or from about 28 μm to about 36 μm, or from about 29 μm to about 36 μm, or from about 30 μm to about 36 μm, or from about 31 μm to about 36 μm, or from about 32 μm to about 36 μm, or from about 33 μm to about 36 μm, or from about 34 μm to about 36 μm, or from about 35 μm to about 36 μm, or from about 25 μm to about 35 μm, or from about 26 μm to about 35 μm, or from about 27 μm to about 35 μm, or from about 28 μm to about 35 μm, or from about 29 μm to about 35 μm, or from about 30 μm to about 35 μm, or from about 31 μm to about 35 μm, or from about 32 μm to about 35 μm, or from about 33 μm to about 35 μm, or from about 34 μm to about 35 μm, or from about 25 μm to about 34 μm, or from about 26 μm to about 34 μm, or from about 27 μm to about 34 μm, or from about 28 μm to about 34 μm, or from about 29 μm to about 34 μm, or from about 30 μm to about 34 μm, or from about 31 μm to about 34 μm, or from about 32 μm to about 34 μm, or from about 33 μm to about 34 μm, or from about 25 μm to about 33 μm, or from about 26 μm to about 33 μm, or from about 27 μm to about 33 μm, or from about 28 μm to about 33 μm, or from about 29 μm to about 33 μm, or from about 30 μm to about 33 μm, or from about 31 μm to about 33 μm, or from about 32 μm to about 33 μm, or from about 25 μm to about 32 μm, or from about 26 μm to about 32 μm, or from about 27 μm to about 32 μm, or from about 28 μm to about 32 μm, or from about 29 μm to about 32 μm, or from about 30 μm to about 32 μm, or from about 31 μm to about 32 μm, or from about 25 μm to about 31 μm, or from about 26 μm to about 31 μm, or from about 27 μm to about 31 μm, or from about 28 μm to about 31 μm, or from about 29 μm to about 31 μm, or from about 30 μm to about 31 μm, or from about 25 μm to about 30 μm, or from about 26 μm to about 30 μm, or from about 27 μm to about 30 μm, or from about 28 μm to about 30 μm, or from about 29 μm to about 30 μm, or from about 25 μm to about 29 μm, or from about 26 μm to about 29 μm, or from about 27 μm to about 29 μm, or from about 28 μm to about 29 μm, or from about 25 μm to about 28 μm, or from about 26 μm to about 28 μm, or from about 27 μm to about 28 μm, or from about 25 μm to about 27 μm, or from about 26 μm to about 27 μm, or from about 25 μm to about 26 μm, or 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, or 70 μm.

The favored abrasion ration value is between 0 and 88 in accordance to the DESAUTELS and LABRECHE 1999 scale. The abrasiveness scale of DESAUTELS and LABRECHE varies as follows for toothpaste: 1) bit abrasive: 0% to 88%; 2) abrasive to medium abrasive: 88% to 100% and 3) very abrasive: >100%.

Specific surface area (SSA), or Brunauer, Emmett and Teller (BET) SSA is a property of solids defined as the total surface area of a material per unit of mass. The particles of treated aragonite calcium carbonate of the present invention may have a specific surface area (in m²/g) of about 2.7 to about 3.1, or about 2.75 to about 3.1, or about 2.8 to about 3.1, or about 2.85 to about 3.1, or about 2.9 to about 3.1, or about 2.95 to about 3.1, or about 3 to about 3.1, or about 3.05 to about 3.1, or about 2.7 to about 3.05, or about 2.75 to about 3.05, or about 2.8 to about 3.05, or about 2.85 to about 3.05, or about 2.9 to about 3.05, or about 2.95 to about 3.05, or about 3 to about 3.05, or about 2.7 to about 3.00, or about 2.75 to about 3.00, or about 2.8 to about 3.00, or about 2.85 to about 3.00, or about 2.9 to about 3.00, or about 2.95 to about 3.00, or about 2.7 to about 2.95, or about 2.75 to about 2.95, or about 2.8 to about 2.95, or about 2.85 to about 2.95, or about 2.9 to about 2.95, or about 2.7 to about 2.90, or about 2.75 to about 2.90, or about 2.8 to about 2.90, or about 2.85 to about 2.90, or about 2.7 to about 2.85, or about 2.75 to about 2.85, or about 2.8 to about 2.85, or about 2.7 to about 2.80, or about 2.75 to about 2.80, or about 2.7 to about 2.75, or 2.7, 2.75, 2.8, 2.85, 2.9, 2.95, 3, 3.05, or 3.1 m²/g.

Crystallinity refers to the degree of structural order in a solid. In a crystal, the atoms or molecules are arranged in a regular, periodic manner. The degree of crystallinity influences the hardness, density, transparency, and diffusion of the solid. The crystallinity of the treated oolitic aragonite used in the present invention may be from about 24% to about 28%, or about 24% to about 27.5%, or about 24% to about 27%, or about 24% to about 26.5%, or about 24% to about 26%, or about 24% to about 25.5%, or about 24% to about 25%, or about 24% to about 24.5%, or about 24.5% to about 28%, or about 24.5% to about 27.5%, or about 24.5% to about 27%, or about 24.5% to about 26.5%, or about 24.5% to about 26%, or about 24.5% to about 25.5%, or about 24.5% to about 25%, or about 25% to about 28%, or about 25% to about 27.5%, or about 25% to about 27%, or about 25% to about 26.5%, or about 25% to about 26%, or about 25% to about 25.5%, or about 25.5% to about 28%, or about 25.5% to about 27.5%, or about 25.5% to about 27%, or about 25.5% to about 26.5%, or about 25.5% to about 26%, or about 26% to about 28%, or about 26% to about 27.5%, or about 26% to about 27%, or about 26% to about 26.5%, or about 26.5% to about 28%, or about 26.5% to about 27.5%, or about 26.5% to about 27%, or about 27% to about 28%, or about 27% to about 27.5%, or about 27.5% to about 28%, or about 24, 24.5, 25, 25.5, 26, 26.5, 27, 27.5, or 28% crystallinity.

The particles of treated aragonite calcium carbonate of the present invention have been treated in mildly acidic conditions, for example in ammonium chloride or ammonium acetate, at pH from about 4.5 to about 5.5, or from about 4.5 to about 5.4, or from about 4.5 to about 5.3, or from about 4.5 to about 5.2, or from about 4.5 to about 5.1, or from about 4.5 to about 5.0, or from about 4.5 to about 4.9, or from about 4.5 to about 4.8, or from about 4.5 to about 4.7, or from about 4.5 to about 4.6, or from about 4.6 to about 5.5, or from about 4.6 to about 5.4, or from about 4.6 to about 5.3, or from about 4.6 to about 5.2, or from about 4.6 to about 5.1, or from about 4.6 to about 5.0, or from about 4.6 to about 4.9, or from about 4.6 to about 4.8, or from about 4.6 to about 4.7, or from about 4.7 to about 5.5, or from about 4.7 to about 5.4, or from about 4.7 to about 5.3, or from about 4.7 to about 5.2, or from about 4.7 to about 5.1, or from about 4.7 to about 5.0, or from about 4.7 to about 4.9, or from about 4.7 to about 4.8, or from about 4.8 to about 5.5, or from about 4.8 to about 5.4, or from about 4.8 to about 5.3, or from about 4.8 to about 5.2, or from about 4.8 to about 5.1, or from about 4.8 to about 5.0, or from about 4.8 to about 4.9, or from about 4.9 to about 5.5, or from about 4.9 to about 5.4, or from about 4.9 to about 5.3, or from about 4.9 to about 5.2, or from about 4.9 to about 5.1, or from about 4.9 to about 5.0, or from about 5.0 to about 5.5, or from about 5.0 to about 5.4, or from about 5.0 to about 5.3, or from about 5.0 to about 5.2, or from about 5.0 to about 5.1, or from about 5.1 to about 5.5, or from about 5.1 to about 5.4, or from about 5.1 to about 5.3, or from about 5.1 to about 5.2, or from about 5.2 to about 5.5, or from about 5.2 to about 5.4, or from about 5.2 to about 5.3, or from about 5.3 to about 5.5, or from about 5.3 to about 5.4, or from about 5.4 to about 5.5, and preferably pH about 4.75, 4.76, 4.77, 4.78, 4.79, 4.80, 4.81, 4.82, 4.83, 4.84, 4.85, 4.86, 4.87, 4.88, 4.89, 4.90, and most preferably 4.86 in order to solubilize unwanted magnesium, ammonia, iron and zinc compounds present in the bone material, and increase the calcium carbonate content of the powder of the present invention. Indeed, the bone powder used in the present invention comprises a high content in calcium; containing at least 95% calcium carbonate, with reduced amounts of magnesium, zinc, iron and ammonia containing derivatives. According to embodiments, the calcium carbonate of the cuttlefish bone powder particles may be at least 95%, at least 95.5%, at least 96%, at least 96.5%, at least 97%, at least 97.5%, at least 98%, at least 98.5%, at least 99%, or from about 95% to 99% (w/w), or from about 95% to 98.5% (w/w), or from about 95% to about 98%, or from about 95% to 97.5% (w/w), or from about 95% to about 97%, or from about 95% to 96.5% (w/w), or from about 95% to about 96%, or from about 95% to 95.5% (w/w), or from about 95.5% to 99% (w/w), or from about 95.5% to 98.5% (w/w), or from about 95.5% to about 98%, or from about 95.5% to 97.5% (w/w), or from about 95.5% to about 97%, or from about 95.5% to 96.5% (w/w), or from about 95.5% to about 96%, or from about 96% to 99% (w/w), or from about 96% to 98.5% (w/w), or from about 96% to about 98%, or from about 96% to 97.5% (w/w), or from about 96% to about 97%, or from about 96% to 96.5% (w/w), or from about 96.5% to 99% (w/w), or from about 96.5% to 98.5% (w/w), or from about 96.5% to about 98%, or from about 96.5% to 97.5% (w/w), or from about 96.5% to about 97%, or from about 97% to 99% (w/w), or from about 97% to 98.5% (w/w), or from about 97% to about 98%, or from about 97% to 97.5% (w/w), or from about 97.5% to 99% (w/w), or from about 97.5% to 98.5% (w/w), or from about 97.5% to about 98%, or from about 98% to 99% (w/w), or from about 98% to 98.5% (w/w), or from about 98.5% to 99% (w/w).

In embodiments, the mildly acidic treatment may be performed with mild acids such as ammonium chloride, ammonium bromide, ammonium acetate, ammonium carbonate, ammonium phosphate, ammonium formate, ammonium malate, triammonium citrate, ammonium tartrate, acetic acid, citric acid, ascorbic acid, tannic acid, boric acids, lactic acid, formic acid, oxalic acid, uric acid, malic acid, tartaric acid, phosphorous acid and the likes. Stronger acids such as hydrochloric acid and phosphoric acids may also be used in dilute conditions that result in mildly acidic treatment of the calcium carbonate.

In embodiment, concentrations of the acids for providing the mild acid treatment will vary according to the acid compound used. For example, for ammonium chloride, the concentration may be from about 0.1 M to about 10 M, preferable about 1.87 M or 2 M.

According to embodiments of the present invention, the treated oolittic aragonite calcium carbonate described above may represent from about 0.1% to about 25% (w/w), or from about 0.1% to about 25% (w/w), or from about 0.1% to about 25% (w/w), or from about 0.1% to about 24%, or from about 0.1% to about 23%, or from about 0.1% to about 22%, or from about 0.1% to about 21%, or from about 0.1% to about 20%, or from about 0.1% to about 19%, or from about 0.1% to about 18%, or from about 0.1% to about 17%, or from about 0.1% to about 16%, or from about 0.1% to about 15%, or from about 0.1% to about 14%, or from about 0.1% to about 13%, or from about 0.1% to about 12%, or from about 0.1% to about 11%, or from about 0.1% to about 10%, or from about 0.1% to about 9%, or from about 0.1% to about 8%, or from about 0.1% to about 7%, or from about 0.1% to about 6%, or from about 0.1% to about 5%, or from about 0.1% to about 4%, or from about 0.1% to about 3%, or from about 0.1% to about 2%, or from about 0.1% to about 1%, or from about 0.1% to about 0.5%, or from about 0.5% to about 25% (w/w), or from about 0.5% to about 25% (w/w), or from about 0.5% to about 25% (w/w), or from about 0.5% to about 24%, or from about 0.5% to about 23%, or from about 0.5% to about 22%, or from about 0.5% to about 21%, or from about 0.5% to about 20%, or from about 0.5% to about 19%, or from about 0.5% to about 18%, or from about 0.5% to about 17%, or from about 0.5% to about 16%, or from about 0.5% to about 15%, or from about 0.5% to about 14%, or from about 0.5% to about 13%, or from about 0.50.5% to about 12%, or from about 0.5% to about 11%, or from about 0.5% to about 10%, or from about 0.5% to about 9%, or from about 0.5% to about 8%, or from about 0.5% to about 7%, or from about 0.5% to about 6%, or from about 0.5% to about 5%, or from about 0.5% to about 4%, or from about 0.5% to about 3%, or from about 0.5% to about 2%, or from about 0.5% to about 1%, or from about 1% to about 25% (w/w), or from about 1% to about 25% (w/w), or from about 1% to about 24%, or from about 1% to about 23%, or from about 1% to about 22%, or from about 1% to about 21%, or from about 1% to about 20%, or from about 1% to about 19%, or from about 1% to about 18%, or from about 1% to about 17%, or from about 1% to about 16%, or from about 1% to about 15%, or from about 1% to about 14%, or from about 1% to about 13%, or from about 1% to about 12%, or from about 1% to about 11%, or from about 1% to about 10%, or from about 1% to about 9%, or from about 1% to about 8%, or from about 1% to about 7%, or from about 1% to about 6%, or from about 1% to about 5%, or from about 1% to about 4%, or from about 1% to about 3%, or from about 1% to about 2%, or from about 2% to about 25% (w/w), or from about 2% to about 24%, or from about 2% to about 23%, or from about 2% to about 22%, or from about 2% to about 21%, or from about 2% to about 20%, or from about 2% to about 19%, or from about 2% to about 18%, or from about 2% to about 17%, or from about 2% to about 16%, or from about 2% to about 15%, or from about 2% to about 14%, or from about 2% to about 13%, or from about 2% to about 12%, or from about 2% to about 11%, or from about 2% to about 10%, or from about 2% to about 9%, or from about 2% to about 8%, or from about 2% to about 7%, or from about 2% to about 6%, or from about 2% to about 5%, or from about 2% to about 4%, or from about 2% to about 3%, or from about 3% to about 25% (w/w), or from about 3% to about 24%, or from about 3% to about 23%, or from about 3% to about 22%, or from about 3% to about 21%, or from about 3% to about 20%, or from about 3% to about 19%, or from about 3% to about 18%, or from about 3% to about 17%, or from about 3% to about 16%, or from about 3% to about 15%, or from about 3% to about 14%, or from about 3% to about 13%, or from about 3% to about 12%, or from about 3% to about 11%, or from about 3% to about 10%, or from about 3% to about 9%, or from about 3% to about 8%, or from about 3% to about 7%, or from about 3% to about 6%, or from about 3% to about 5%, or from about 3% to about 4%, or from about 4% to about 25% (w/w), or from about 4% to about 24%, or from about 4% to about 23%, or from about 4% to about 22%, or from about 4% to about 21%, or from about 4% to about 20%, or from about 4% to about 19%, or from about 4% to about 18%, or from about 4% to about 17%, or from about 4% to about 16%, or from about 4% to about 15%, or from about 4% to about 14%, or from about 4% to about 13%, or from about 4% to about 12%, or from about 4% to about 11%, or from about 4% to about 10%, or from about 4% to about 9%, or from about 4% to about 8%, or from about 4% to about 7%, or from about 4% to about 6%, or from about 4% to about 5%, or from about 5% to about 25% (w/w), or from about 5% to about 24%, or from about 5% to about 23%, or from about 5% to about 22%, or from about 5% to about 21%, or from about 5% to about 20%, or from about 5% to about 19%, or from about 5% to about 18%, or from about 5% to about 17%, or from about 5% to about 16%, or from about 5% to about 15%, or from about 5% to about 14%, or from about 5% to about 13%, or from about 5% to about 12%, or from about 5% to about 11%, or from about 5% to about 10%, or from about 5% to about 9%, or from about 5% to about 8%, or from about 5% to about 7%, or from about 5% to about 6%, or from about 6% to about 25% (w/w), or from about 6% to about 24%, or from about 6% to about 23%, or from about 6% to about 22%, or from about 6% to about 21%, or from about 6% to about 20%, or from about 6% to about 19%, or from about 6% to about 18%, or from about 6% to about 17%, or from about 6% to about 16%, or from about 6% to about 15%, or from about 6% to about 14%, or from about 6% to about 13%, or from about 6% to about 12%, or from about 6% to about 11%, or from about 6% to about 10%, or from about 6% to about 9%, or from about 6% to about 8%, or from about 6% to about 7%, or from about 7% to about 25% (w/w), or from about 7% to about 24%, or from about 7% to about 23%, or from about 7% to about 22%, or from about 7% to about 21%, or from about 7% to about 20%, or from about 7% to about 19%, or from about 7% to about 18%, or from about 7% to about 17%, or from about 7% to about 16%, or from about 7% to about 15%, or from about 7% to about 14%, or from about 7% to about 13%, or from about 7% to about 12%, or from about 7% to about 11%, or from about 7% to about 10%, or from about 7% to about 9%, or from about 7% to about 8%, or from about 8% to about 25% (w/w), or from about 8% to about 24%, or from about 8% to about 23%, or from about 8% to about 22%, or from about 8% to about 21%, or from about 8% to about 20%, or from about 8% to about 19%, or from about 8% to about 18%, or from about 8% to about 17%, or from about 8% to about 16%, or from about 8% to about 15%, or from about 8% to about 14%, or from about 8% to about 13%, or from about 8% to about 12%, or from about 8% to about 11%, or from about 8% to about 10%, or from about 8% to about 9%, or from about 9% to about 25% (w/w), or from about 9% to about 24%, or from about 9% to about 23%, or from about 9% to about 22%, or from about 9% to about 21%, or from about 9% to about 20%, or from about 9% to about 19%, or from about 9% to about 18%, or from about 9% to about 17%, or from about 9% to about 16%, or from about 9% to about 15%, or from about 9% to about 14%, or from about 9% to about 13%, or from about 9% to about 12%, or from about 9% to about 11%, or from about 9% to about 10%, or from about 10% to about 25% (w/w), or from about 10% to about 24%, or from about 10% to about 23%, or from about 10% to about 22%, or from about 10% to about 21%, or from about 10% to about 20%, or from about 10% to about 19%, or from about 10% to about 18%, or from about 10% to about 17%, or from about 10% to about 16%, or from about 10% to about 15%, or from about 10% to about 14%, or from about 10% to about 13%, or from about 10% to about 12%, or from about 10% to about 11%, or from about 11% to about 25% (w/w), or from about 11% to about 24%, or from about 11% to about 23%, or from about 11% to about 22%, or from about 11% to about 21%, or from about 11% to about 20%, or from about 11% to about 19%, or from about 11% to about 18%, or from about 11% to about 17%, or from about 11% to about 16%, or from about 11% to about 15%, or from about 11% to about 14%, or from about 11% to about 13%, or from about 11% to about 12%, or from about 12% to about 25% (w/w), or from about 12% to about 24%, or from about 12% to about 23%, or from about 12% to about 22%, or from about 12% to about 21%, or from about 12% to about 20%, or from about 12% to about 19%, or from about 12% to about 18%, or from about 12% to about 17%, or from about 12% to about 16%, or from about 12% to about 15%, or from about 12% to about 14%, or from about 12% to about 13%, or from about 13% to about 25% (w/w), or from about 13% to about 24%, or from about 13% to about 23%, or from about 13% to about 22%, or from about 13% to about 21%, or from about 13% to about 20%, or from about 13% to about 19%, or from about 13% to about 18%, or from about 13% to about 17%, or from about 13% to about 16%, or from about 13% to about 15%, or from about 13% to about 14%, or from about 14% to about 25% (w/w), or from about 14% to about 24%, or from about 14% to about 23%, or from about 14% to about 22%, or from about 14% to about 21%, or from about 14% to about 20%, or from about 14% to about 19%, or from about 14% to about 18%, or from about 14% to about 17%, or from about 14% to about 16%, or from about 14% to about 15%, or from about 15% to about 25% (w/w), or from about 15% to about 24%, or from about 15% to about 23%, or from about 15% to about 22%, or from about 15% to about 21%, or from about 15% to about 20%, or from about 15% to about 19%, or from about 15% to about 18%, or from about 15% to about 17%, or from about 15% to about 16%, or from about 16% to about 25% (w/w), or from about 16% to about 24%, or from about 16% to about 23%, or from about 16% to about 22%, or from about 16% to about 21%, or from about 16% to about 20%, or from about 16% to about 19%, or from about 16% to about 18%, or from about 16% to about 17%, or from about 17% to about 25% (w/w), or from about 17% to about 24%, or from about 17% to about 23%, or from about 17% to about 22%, or from about 17% to about 21%, or from about 17% to about 20%, or from about 17% to about 19%, or from about 17% to about 18%, or from about 18% to about 25% (w/w), or from about 18% to about 24%, or from about 18% to about 23%, or from about 18% to about 22%, or from about 18% to about 21%, or from about 18% to about 20%, or from about 18% to about 19%, or from about 19% to about 25% (w/w), or from about 19% to about 24%, or from about 19% to about 23%, or from about 19% to about 22%, or from about 19% to about 21%, or from about 19% to about 20%, or from about 20% to about 25% (w/w), or from about 20% to about 24%, or from about 20% to about 23%, or from about 20% to about 22%, or from about 20% to about 21%, or from about 21% to about 25% (w/w), or from about 21% to about 24%, or from about 21% to about 23%, or from about 21% to about 22%, or from about 22% to about 25% (w/w), or from about 22% to about 24%, or from about 22% to about 23%, or from about 23% to about 25% (w/w), or from about 23% to about 24%, or from about 24% to about 25% (w/w), or 0.1, 0.5, 1, 2, 3, 4, 5, 6, 7, 8, 9, 10, 11, 12, 13, 14, 15, 16, 17, 18, 19, 20, 21, 22, 23, 24, or 25% of the composition. Preferred embodiments may comprise from about 4.3%, 5%, 6.3%, 8.5%, 10%, 11.2%, 11.5, 12%, 15%, and 15.2% w/w.

In embodiments, the composition of the present invention comprises a fluoride compound suitable to provide beneficial fluoride treatment to teeth. In embodiments, the fluoride compound may be sodium fluoride (NaF), stannous fluoride (SnF₂), sodium monofluorophosphate (MFP—Na₂PO₃F), or combinations thereof. The fluoride compounds may be present at concentrations of fluoride of from about 800 ppm to about 5000 ppm fluoride, or from about 1000 ppm to about 1500 ppm fluoride, or 800, 1000, 1500, or 5000 ppm fluoride.

The composition of the present invention may comprise a number of ingredients, which include:

### Abrasives

According to an embodiment, the oral care composition of the present invention may contain a second dental abrasive in addition to the treated aragonite calcium carbonate (CaCO₃) particles used in the present invention. Preferably, the abrasive is chosen from colloidal calcium or colloidal silica. Suitable abrasives include hydrated silica and sodium bicarbonate (NaHCO₃). Other suitable abrasives include but are not limited to aluminum hydroxide (Al(OH)₃), calcium carbonate (CaCO₃), various calcium hydrogen phosphates (CaHPO₄·2H₂O, or anhydrous), various silicas (such as fumed silica, precipitated silica) and zeolites, and hydroxyapatite (Ca₅(PO₄)₃OH). Abrasive are insoluble particles that help remove tartar (plaque) from the teeth and help remove dead cells from the skin. In toothpaste systems, the abrasive silica was shown to be the principal tooth cleaning and abrasive agent. In embodiments, the second dental abrasive may be a sodium bicarbonate (NaHCO₃), a colloidal silica, or a combination thereof.

According to an embodiment second dental abrasive may constitute from about 0.100% to about 30%, or from about 1% to about 30%, or from about 2% to about 30%, or from about 3% to about 30%, or from about 4% to about 30%, or from about 5% to about 30%, or from about 6% to about 30%, or from about 7% to about 30%, or from about 8% to about 30%, or from about 9% to about 30%, or from about 10% to about 30%, or from about 11% to about 30%, or from about 12% to about 30%, or from about 13% to about 30%, or from about 14% to about 30%, or from about 15% to about 30%, or from about 16% to about 30%, or from about 17% to about 30%, or from about 18% to about 30%, or from about 19% to about 30%, or from about 20% to about 30%, or from about 21% to about 30%, or from about 22% to about 30%, or from about 23% to about 30%, or from about 24% to about 30%, or from about 25% to about 30%, or from about 26% to about 30%, or from about 27% to about 30%, or from about 28% to about 30%, or from about 29% to about 30%, or 0.100% to about 20%, or from about 1% to about 20%, or from about 2% to about 20%, or from about 3% to about 20%, or from about 4% to about 20%, or from about 5% to about 20%, or from about 6% to about 20%, or from about 7% to about 20%, or from about 8% to about 20%, or from about 9% to about 20%, or from about 10% to about 20%, or from about 11% to about 20%, or from about 12% to about 20%, or from about 13% to about 20%, or from about 14% to about 20%, or from about 15% to about 20%, or from about 16% to about 20%, or from about 17% to about 20%, or from about 18% to about 20%, or from about 19% to about 20%, or 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% (w/w) of the composition. According to another embodiment, the colloidal silica may be from about 0.1% to about 20% (w/w) of the composition. According to another embodiment, the sodium bicarbonate (NaHCO₃) may be from about 0.02% to about 0.75% (w/w) of the composition.

### Thickening Agents

According to an embodiment, the personal care composition of the present invention may contain a thickening agent.

Thickening agents, or thickeners, are substances which increase the viscosity of a solution or liquid/solid mixture without substantially modifying its other properties; although most frequently applied to foods where the target property is taste, the term also is applicable to paints, inks, explosives, etc. Thickeners may also be referred to as “natural gums”. Thickeners may also improve the suspension of other ingredients or emulsions which increases the stability of the product. Thickening agents are often regulated as food additives and as cosmetics and personal hygiene product ingredients. Some thickening agents are gelling agents (gellants), forming a gel, dissolving in the liquid phase as a colloid mixture that forms a weakly cohesive internal structure. Examples of suitable thickeners include but are not limited to natural gums obtained from seaweeds, such as agar (E406), alginic acid (E400) and Sodium alginate (E401), potassium alginate, ammonium alginate, calcium alginate, carrageenan (E407); natural gums obtained from non-marine botanical resources, acacia gum, gum arabic (E414), gum ghatti, gum tragacanth (E413), karaya gum (E416), guar gum (E412), locust bean gum (E410), beta-glucan, chicle gum, dammar gum, Glucomannan (E425), mastic gum, psyllium seed husks, spruce gum, tara gum (E417); natural gums produced by bacterial fermentation: gellan gum (E418), Xanthan gum (E415).

Also included are starches, pectins, carboxymethyl celluloses, hydroxypropyl celluloses, methyl cellulose and gelatin. Cellulose gum is the common name for carboxymethylcellulose, or CMC. Its emulsifying properties make it especially useful for products with ingredients that tend to separate, such as yogurt and jellies. Its ability to bind water makes it especially useful for diet foods, which tend to substitute water or other liquids for fat. Cellulose gum also improves texture, so it is a common ingredient in ice cream and frosting, products in which smoothness is a mark of quality. Beer manufacturers also use cellulose gum to stabilize beer foam. These same properties are useful for some pharmaceutical products that tend to separate over time, such as toothpaste. In the cosmetics industry, cellulose gum appears in bath products, makeup, shaving gels and hair products. According to an embodiment, the preferred thickening agents include but are not limited to xanthan gum, carboxymethylcellulose, and guar gum.

According to another embodiment, the thickening agent may be present in the formulation in about from about 0.1% to about 66% (w/w), or from about 0.5% to about 66% (w/w), or from about 1% to about 66% (w/w), 2% to about 66% (w/w), or from about 5% to about 66% (w/w), or from about 10% to about 66% (w/w), or from about 15% to about 66% (w/w), or from about 20% to about 66% (w/w), or from about 25% to about 66% (w/w), or from about 30% to about 66% (w/w), or from about 35% to about 66% (w/w), or from about 40% to about 66% (w/w), or from about 45% to about 66% (w/w), or from about 50% to about 66% (w/w), or from about 55% to about 66% (w/w), or from about 60% to about 66% (w/w), or from about 2% to about 60% (w/w), or from about 5% to about 60% (w/w), or from about 10% to about 60% (w/w), or from about 15% to about 60% (w/w), or from about 20% to about 60% (w/w), or from about 25% to about 60% (w/w), or from about 30% to about 60% (w/w), or from about 35% to about 60% (w/w), or from about 40% to about 60% (w/w), or from about 45% to about 60% (w/w), or from about 50% to about 60% (w/w), or from about 55% to about 60% (w/w), or from about 2% to about 55% (w/w), or from about 5% to about 55% (w/w), or from about 10% to about 55% (w/w), or from about 15% to about 55% (w/w), or from about 20% to about 55% (w/w), or from about 25% to about 55% (w/w), or from about 30% to about 55% (w/w), or from about 35% to about 55% (w/w), or from about 40% to about 55% (w/w), or from about 45% to about 55% (w/w), or from about 50% to about 55% (w/w), or from about 2% to about 50% (w/w), or from about 5% to about 50% (w/w), or from about 10% to about 50% (w/w), or from about 15% to about 50% (w/w), or from about 20% to about 50% (w/w), or from about 25% to about 50% (w/w), or from about 30% to about 50% (w/w), or from about 35% to about 50% (w/w), or from about 40% to about 50% (w/w), or from about 45% to about 50% (w/w), or from about 2% to about 45% (w/w), or from about 5% to about 45% (w/w), or from about 10% to about 45% (w/w), or from about 15% to about 45% (w/w), or from about 20% to about 45% (w/w), or from about 25% to about 45% (w/w), or from about 30% to about 45% (w/w), or from about 35% to about 45% (w/w), or from about 40% to about 45% (w/w), or from about 2% to about 40% (w/w), or from about 5% to about 40% (w/w), or from about 10% to about 40% (w/w), or from about 15% to about 40% (w/w), or from about 20% to about 40% (w/w), or from about 25% to about 40% (w/w), or from about 30% to about 40% (w/w), or from about 35% to about 40% (w/w), or from about 2% to about 35% (w/w), or from about 5% to about 35% (w/w), or from about 10% to about 35% (w/w), or from about 15% to about 35% (w/w), or from about 20% to about 35% (w/w), or from about 25% to about 35% (w/w), or from about 30% to about 35% (w/w), or from about 2% to about 30% (w/w), or from about 5% to about 30% (w/w), or from about 10% to about 30% (w/w), or from about 15% to about 30% (w/w), or from about 20% to about 30% (w/w), or from about 25% to about 30% (w/w), or from about 2% to about 25% (w/w), or from about 5% to about 25% (w/w), or from about 10% to about 25% (w/w), or from about 15% to about 25% (w/w), or from about 20% to about 25% (w/w), or from about 2% to about 20% (w/w), or from about 5% to about 20% (w/w), or from about 10% to about 20% (w/w), or from about 15% to about 20% (w/w), or from about 2% to about 15% (w/w), or from about 5% to about 15% (w/w), or from about 10% to about 15% (w/w), or from about 2% to about 10% (w/w), or from about 5% to about 10% (w/w), or from about 2% to about 5% (w/w), or 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 387, 39, 40, 41, 42, 43, 44, 45, 46, 47, 48, 49, 50, 51, 52, 53, 54, 55, 56, 57, 58, 59, 60, 61, 62, 63, 64, 65, or 66% (w/w). According to an embodiment, the concentration is about 0.5% (w/w).

### Humectants

According to another embodiment, the composition of the present invention may further comprise a humectant. Humectants are substance used to keep things moist. When used as a food additive, the humectant has the effect of keeping the foodstuff moist. Humectants are also found in many cosmetic products where moisturization is desired, including treatments such as moisturizing hair conditioners and also commonly used in body lotions. Examples of humectants include but are not limited to propylene glycol, as well as hexylene glycol and butylene glycol, glyceryl triacetate, vinyl alcohol, neoagarobiose, sugar polyols such as glycerol, sorbitol, xylitol and maltitol, polymeric polyols like polydextrose, polyethylene glycol, polypropylene glycol, and poly(tetramethylene ether) glycol, quillaia, lactic acid, urea, glycerin, aloe vera gel, MP Diol, alpha hydroxy acids like lactic acid, and honey. According to another embodiment, the preferred humectant may glycerol, according to another preferred embodiment, the preferred humectants may be glycerol, xylitol, sorbitol, or a combination thereof.

According to another embodiment of the present invention, the humectant may be from about 2% to about 45% (w/w), or from about 2% to about 40% (w/w), or from about 2% to about 35% (w/w), or from about 2% to about 30% (w/w), or from about 2% to about 25% (w/w), or from about 2% to about 20% (w/w), or from about 2% to about 15% (w/w), or from about 2% to about 10% (w/w), or from about 2% to about 9% (w/w), or from about 2% to about 8% (w/w), or from about 2% to about 7% (w/w), or from about 2% to about 6% (w/w), or from about 2% to about 5% (w/w), or from about 2% to about 4% (w/w), or from about 2% to about 3% (w/w), or from about 3% to about 5% (w/w), or from about 3% to about 4% (w/w), or from about 4% to about 5% (w/w), or about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45% (w/w) of the composition.

### Emulsifier

According to an embodiment, the composition of the present invention may further comprise an emulsifier. An emulsifier is a substance that stabilizes an emulsion by increasing its kinetic stability. According to an embodiment, the emulsifier may be a lecithin, a vegetal pulp powder (such as citrus pulp powder, baobab pulp powder, mango pulp powder, tomato pulp powder, pumpkin pulp powder, guava pulp powder, papaya pulp powder and beet pulp powder), sodium citrate (e.g. trisodium citrate) and citric acid. The preferred emulsifier is sodium citrate.

According to another embodiment of the present invention, the emulsifier may be from about 1% to about 10%, or from about 2% to about 10%, or from about 3% to about 10%, or from about 4% to about 10%, or from about 4% to about 9%, or from about 4% to about 8%, or from about 4% to about 7%, or from about 4% to about 6%, or from about 4% to about 5%, or from about 5% to about 10%, or from about 5% to about 9%, or from about 5% to about 8%, or from about 5% to about 7%, or from about 5% to about 6%, or from about 6% to about 10%, or from about 6% to about 9%, or from about 6% to about 8%, or from about 6% to about 7%, or from about 7% to about 10%, or from about 7% to about 9%, or from about 7% to about 8%, or from about 8% to about 10%, or from about 8% to about 9%, or from about 9% to about 10% (w/w), or about 1%, 2%, 3%, 4%, 5%, 6%, 7%, 8%, 9% 10% (w/w) of the composition.

### Surfactants

According to an embodiment, the composition of the present invention may further comprise a surfactant. Surfactants are often, but always included in toothpaste and other oral care compositions. For example, toothpastes may contain sodium lauryl sulfate (SLS, also known as sodium dodecyl sulfate, SDS) or related surfactants (detergents). SLS is found in many other personal care products, as well, such as shampoo, and is mainly a foaming agent, which enables uniform distribution of toothpaste, improving its cleansing power. Other suitable surfactants include, but are not limited to ammonium lauryl sulfate, sodium N-lauryl sarcosinate (also known as sodium sarcosinate, and sodium lauryl sarcosinate) and sodium lauryl sulfoacetate.

Surfactants (detergents) also help clean the teeth and provide foam that helps to carry away debris. Moreover, lauryl sulfates have significant anti-bacterial properties, and they can penetrate and dissolve plaque.

According to an embodiment, the surfactant may be from about 0.5% to about 3%, or from about 1% to about 3% (w/w), or from about 2% to about 3% (w/w), or from about 1% to about 2% (w/w), or from about 2% to about 3%, or 0.5%, 1%, 2%, 3% (w/w) of surfactant.

### pH Regulator

According to an embodiment, the compositions of the present invention may contain a pH regulator. The product pH influences its stability and quality. When the pH is very acid, demineralization is favored, but if it is too basic, calcareous (tartar) deposits on the tooth can become important. Thus, the pH is preferably close to neutral pH, for example from about 6 to about 8, or from about 6.5, to about 7.5, or from about 6.75 to about 7.25, or about 7.0. The measured pH of the product is close to 6.8, or more specifically 6.78.

In embodiment, the pH regulator is an acid or a base which when added to the formulation stabilizes the pH at a desired level suitable for the oral care formulation of the present invention. Suitable pH regulator include but are not limited to citric acid and its derivatives, phosphoric acid and its derivatives, trisodium phosphate, sodium citrate, lactic acid, bicarbonic acid. The pH regulator may be present in concentrations of about 0.1% to about 0.28% (w/w), or from about 0.1% to about 0.25%, or from about 0.1% to about 0.2%, or from about 0.1% to about 0.15%, or from about 0.1% to about 0.12%, or about 0.12% to about 0.28%, or from about 0.12% to about 0.25%, or from about 0.12% to about 0.2%, or from about 0.12% to about 0.15%, or about 0.15% to about 0.28%, or from about 0.15% to about 0.25%, or from about 0.15% to about 0.2%, or about 0.2% to about 0.28%, or from about 0.2% to about 0.25%, or about 0.25% to about 0.28% (w/w), or about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, of the composition.

### Preservative

According to an embodiment, the compositions of the present invention may contain preservative agent. According to an embodiment, the preservative agent may sometime also act as an active antimicrobial agent for having an active role in the use of the composition.

Microorganisms can feed on humectants and thickening agents and ingredients to restrict their growth may be present in toothpaste. Generally, this is accomplished through minimal water and use of preservatives in the formulation. The most common preservatives in toothpaste are sorbitan sesquioleate derivatives, sodium benzoate, potassium sorbate, and benzoic acid. However, the compositions of the present invention may also be formulated with natural ingredients with preservative qualities or non-synthetic versions of common preservatives. Examples of natural products having preservative qualities include but is not limited to eucalyptus extract, essential oil having natural antimicrobial properties, such as eucalyptus oil, thyme oil, oregano oil, lemon oil, orange oil, and the likes, as well as natural antimicrobial agents such as thymol, carvacrol, eugenol, eucalyptol, menthol, etc., which are contained in these essential oils, or may be provided as isolated compounds. The composition may contain from about 0.2% to about 2% w/w, or about 0.3% to about 2% w/w, or about 0.4% to about 2% w/w, or about 0.5% to about 2% w/w, or about 0.6% to about 2% w/w, or about 0.7% to about 2% w/w, or about 0.8% to about 2% w/w, or about 0.9% to about 2% w/w, or about 1% to about 2% w/w, or about 1.1% to about 2% w/w, or about 1.2% to about 2% w/w, or about 1.3% to about 2% w/w, or about 1.4% to about 2% w/w, or about 1.5% to about 2% w/w, or about 1.6% to about 2% w/w, or about 1.7% to about 2% w/w, or about 1.8% to about 2% w/w, or about 1.9% to about 2% w/w, or about 0.2% to about 1.9% w/w, or about 0.3% to about 1.9% w/w, or about 0.4% to about 1.9% w/w, or about 0.5% to about 1.9% w/w, or about 0.6% to about 1.9% w/w, or about 0.7% to about 1.9% w/w, or about 0.8% to about 1.9% w/w, or about 0.9% to about 1.9% w/w, or about 1% to about 1.9% w/w, or about 1.1% to about 1.9% w/w, or about 1.2% to about 1.9% w/w, or about 1.3% to about 1.9% w/w, or about 1.4% to about 1.9% w/w, or about 1.5% to about 1.9% w/w, or about 1.6% to about 1.9% w/w, or about 1.7% to about 1.9% w/w, or about 1.8% to about 1.9% w/w, or about 0.2% to about 1.8% w/w, or about 0.3% to about 1.8% w/w, or about 0.4% to about 1.8% w/w, or about 0.5% to about 1.8% w/w, or about 0.6% to about 1.8% w/w, or about 0.7% to about 1.8% w/w, or about 0.8% to about 1.8% w/w, or about 0.9% to about 1.8% w/w, or about 1% to about 1.8% w/w, or about 1.1% to about 1.8% w/w, or about 1.2% to about 1.8% w/w, or about 1.3% to about 1.8% w/w, or about 1.4% to about 1.8% w/w, or about 1.5% to about 1.8% w/w, or about 1.6% to about 1.8% w/w, or about 1.7% to about 1.8% w/w, or about 0.2% to about 1.7% w/w, or about 0.3% to about 1.7% w/w, or about 0.4% to about 1.7% w/w, or about 0.5% to about 1.7% w/w, or about 0.6% to about 1.7% w/w, or about 0.7% to about 1.7% w/w, or about 0.8% to about 1.7% w/w, or about 0.9% to about 1.7% w/w, or about 1% to about 1.7% w/w, or about 1.1% to about 1.7% w/w, or about 1.2% to about 1.7% w/w, or about 1.3% to about 1.7% w/w, or about 1.4% to about 1.7% w/w, or about 1.5% to about 1.7% w/w, or about 1.6% to about 1.7% w/w, or about 0.2% to about 1.6% w/w, or about 0.3% to about 1.6% w/w, or about 0.4% to about 1.6% w/w, or about 0.5% to about 1.6% w/w, or about 0.6% to about 1.6% w/w, or about 0.7% to about 1.6% w/w, or about 0.8% to about 1.6% w/w, or about 0.9% to about 1.6% w/w, or about 1% to about 1.6% w/w, or about 1.1% to about 1.6% w/w, or about 1.2% to about 1.6% w/w, or about 1.3% to about 1.6% w/w, or about 1.4% to about 1.6% w/w, or about 1.5% to about 1.6% w/w, or about 0.2% to about 1.5% w/w, or about 0.3% to about 1.5% w/w, or about 0.4% to about 1.5% w/w, or about 0.5% to about 1.5% w/w, or about 0.6% to about 1.5% w/w, or about 0.7% to about 1.5% w/w, or about 0.8% to about 1.5% w/w, or about 0.9% to about 1.5% w/w, or about 1% to about 1.5% w/w, or about 1.1% to about 1.5% w/w, or about 1.2% to about 1.5% w/w, or about 1.3% to about 1.5% w/w, or about 1.4% to about 1.5% w/w, or about 0.2% to about 1.4% w/w, or about 0.3% to about 1.4% w/w, or about 0.4% to about 1.4% w/w, or about 0.5% to about 1.4% w/w, or about 0.6% to about 1.4% w/w, or about 0.7% to about 1.4% w/w, or about 0.8% to about 1.4% w/w, or about 0.9% to about 1.4% w/w, or about 1% to about 1.4% w/w, or about 1.1% to about 1.4% w/w, or about 1.2% to about 1.4% w/w, or about 1.3% to about 1.4% w/w, or about 0.2% to about 1.3% w/w, or about 0.3% to about 1.3% w/w, or about 0.4% to about 1.3% w/w, or about 0.5% to about 1.3% w/w, or about 0.6% to about 1.3% w/w, or about 0.7% to about 1.3% w/w, or about 0.8% to about 1.3% w/w, or about 0.9% to about 1.3% w/w, or about 1% to about 1.3% w/w, or about 1.1% to about 1.3% w/w, or about 1.2% to about 1.3% w/w, or about 0.2% to about 1.2% w/w, or about 0.3% to about 1.2% w/w, or about 0.4% to about 1.2% w/w, or about 0.5% to about 1.2% w/w, or about 0.6% to about 1.2% w/w, or about 0.7% to about 1.2% w/w, or about 0.8% to about 1.2% w/w, or about 0.9% to about 1.2% w/w, or about 1% to about 1.2% w/w, or about 1.1% to about 1.2% w/w, or about 0.2% to about 1.1% w/w, or about 0.3% to about 1.1% w/w, or about 0.4% to about 1.1% w/w, or about 0.5% to about 1.1% w/w, or about 0.6% to about 1.1% w/w, or about 0.7% to about 1.1% w/w, or about 0.8% to about 1.1% w/w, or about 0.9% to about 1.1% w/w, or about 1% to about 1.1% w/w, or about 0.2% to about 1.0% w/w, or about 0.3% to about 1.0% w/w, or about 0.4% to about 1.0% w/w, or about 0.5% to about 1.0% w/w, or about 0.6% to about 1.0% w/w, or about 0.7% to about 1.0% w/w, or about 0.8% to about 1.0% w/w, or about 0.9% to about 1.0% w/w, or about 0.2% to about 0.9% w/w, or about 0.3% to about 0.9% w/w, or about 0.4% to about 0.9% w/w, or about 0.5% to about 0.9% w/w, or about 0.6% to about 0.9% w/w, or about 0.7% to about 0.9% w/w, or about 0.8% to about 0.9% w/w, or about 0.2% to about 0.8% w/w, or about 0.3% to about 0.8% w/w, or about 0.4% to about 0.8% w/w, or about 0.5% to about 0.8% w/w, or about 0.6% to about 0.8% w/w, or about 0.7% to about 0.8% w/w, or about 0.2% to about 0.7% w/w, or about 0.3% to about 0.7% w/w, or about 0.4% to about 0.7% w/w, or about 0.5% to about 0.7% w/w, or about 0.6% to about 0.7% w/w, or about 0.2% to about 0.6% w/w, or about 0.3% to about 0.6% w/w, or about 0.4% to about 0.6% w/w, or about 0.5% to about 0.6% w/w, or about 0.2% to about 0.5% w/w, or about 0.3% to about 0.5% w/w, or about 0.4% to about 0.5% w/w, or about 0.2% to about 0.5% w/w, or about 0.3% to about 0.5% w/w, or about 0.4% to about 0.5% w/w, or about 0.2% to about 0.4% w/w, or about 0.3% to about 0.4% w/w, or about 0.2% to about 0.3% w/w, or about 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, and preferably about 0.5% w/w preservative.

### Solvents

According to another embodiment of the present invention, the compositions may comprise suitable solvents to formulate the compositions as mouthwashes, for example. Suitable solvents include but are not limited to water, ethanol, isopropanol, sorbitol and glycerol.

According to an embodiment, the composition may contain from about 40% to about 99% w/w, or about 45% to about 99% w/w, or about 50% to about 99% w/w, or about 55% to about 99% w/w, or about 60% to about 99% w/w, or about 65% to about 99% w/w, or about 70% to about 99% w/w, or about 75% to about 99% w/w, or about 80% to about 99% w/w, or about 85% to about 99% w/w, or about 90% to about 99% w/w, or about 95% to about 99% w/w, or about 40% to about 95% w/w, or about 45% to about 95% w/w, or about 50% to about 95% w/w, or about 55% to about 95% w/w, or about 60% to about 95% w/w, or about 65% to about 95% w/w, or about 70% to about 95% w/w, or about 75% to about 95% w/w, or about 80% to about 95% w/w, or about 85% to about 95% w/w, or about 90% to about 95% w/w, or about 40% to about 90% w/w, or about 45% to about 90% w/w, or about 50% to about 90% w/w, or about 55% to about 90% w/w, or about 60% to about 90% w/w, or about 65% to about 90% w/w, or about 70% to about 90% w/w, or about 75% to about 90% w/w, or about 80% to about 90% w/w, or about 85% to about 90% w/w, or about 40% to about 85% w/w, or about 45% to about 85% w/w, or about 50% to about 85% w/w, or about 55% to about 85% w/w, or about 60% to about 85% w/w, or about 65% to about 85% w/w, or about 70% to about 85% w/w, or about 75% to about 85% w/w, or about 80% to about 85% w/w, or about 40% to about 80% w/w, or about 45% to about 80% w/w, or about 50% to about 80% w/w, or about 55% to about 80% w/w, or about 60% to about 80% w/w, or about 65% to about 80% w/w, or about 70% to about 80% w/w, or about 75% to about 80% w/w, or about 40% to about 75% w/w, or about 45% to about 75% w/w, or about 50% to about 75% w/w, or about 55% to about 75% w/w, or about 60% to about 75% w/w, or about 65% to about 75% w/w, or about 70% to about 75% w/w, or about 40% to about 70% w/w, or about 45% to about 70% w/w, or about 50% to about 70% w/w, or about 55% to about 70% w/w, or about 60% to about 70% w/w, or about 65% to about 70% w/w, or about 40% to about 65% w/w, or about 45% to about 65% w/w, or about 50% to about 65% w/w, or about 55% to about 65% w/w, or about 60% to about 65% w/w, or about 40% to about 60% w/w, or about 45% to about 60% w/w, or about 50% to about 60% w/w, or about 55% to about 60% w/w, or about 40% to about 55% w/w, or about 45% to about 55% w/w, or about 50% to about 55% w/w, or about 40% to about 50% w/w, or about 45% to about 50% w/w, or about 40% to about 45% w/w, or 40%, 45%, 50%, 55%, 60%, 65%, 70%, 75%, 80%, 85%, 90%, 95% or 99% (w/w) of the solvent.

### Antimicrobial Agents

Antimicrobial agents that are useful in the present invention are the so-called “natural” antimicrobial actives. Such antimicrobial agents include natural essential oils and the individual antimicrobial compounds comprised in these oils. These actives derive their names from their natural occurrence in plants. Essential oils include oils derived from herbs, flowers, trees, and other plants. Such oils are typically present as tiny droplets between the plant's cells and can be extracted by several methods known to those of skill in the art (e.g., steam distillation, enfleurage (i.e., extraction using fat(s)), maceration, solvent extraction, or mechanical pressing). Essential oils are typically named by the plant or vegetable in which the oil is found. For example, rose oil or peppermint oil is derived from rose or peppermint plants, respectively. Non-limiting examples of essential oils that can be used in the context of the present invention include oils of anise, lemon oil, orange oil, oregano, rosemary oil, wintergreen oil, thyme oil, lavender oil, clove oil, hops, tea tree oil, citronella oil, wheat oil, barley oil, lemongrass oil, cedar leaf oil, cedar wood oil, cinnamon oil, fleagrass oil, geranium oil, sandalwood oil, violet oil, cranberry oil, eucalyptus oil, vervain oil, peppermint oil, gum benzoin, basil oil, fennel oil, fir oil, balsam oil, menthol, ocmea origanum oil, Hydastis carradensis oil, Berberidaceae daceae oil, Ratanhiae and Curcuma longa oil, sesame oil, macadamia nut oil, evening primrose oil, Spanish sage oil, Spanish rosemary oil, coriander oil, thyme oil, pimento berries oil, rose oil, bergamot oil, rosewood oil, chamomile oil, sage oil, clary sage oil, cypress oil, sea fennel oil, frankincense oil, ginger oil, grapefruit oil, jasmine oil, juniper oil, lime oil, mandarin oil, marjoram oil, myrrh oil, neroli oil, patchouli oil, pepper oil, black pepper oil, petitgrain oil, pine oil, rose otto oil, spearmint oil, spikenard oil, vetiver oil, or ylang ylang. Other essential oils known to those of skill in the art are also contemplated as being useful within the context of the present invention (e.g., International Cosmetic Ingredient Dictionary, 10th edition, 2004, which is incorporated by reference). Also included in this class of essential oils are the key chemical components of the plant oils that have been found to provide the antimicrobial benefit (e.g., antimicrobial phenolic compounds).

The antimicrobial phenolic compounds of natural origin as used in the present invention can be synthetically made by known methods within the capacity of a skilled technician or can be obtained from plant oil extracts. In an embodiment of the present invention, the phenolic compounds of natural origin are obtained from plant extracts. In a further embodiment of the present invention, the phenolic compounds of natural origin are commercially available. In yet further embodiments of the present invention, the phenolic compounds of natural origin comprise carvacrol, thymol, eugenol, eucalyptol, menthol, etc.

In an embodiment, the disinfectant formulations of the present invention comprise thymol, carvacrol or mixtures thereof. In a further embodiment, the disinfectant formulations of the present invention comprise one or more natural essential oils enriched in thymol, carvacrol or mixtures of thymol and carvacrol.

The compositions of the present inventions may contain from about 0.01% to about 10% (w/w), or from about 0.01% to about 9% (w/w), or from about 0.01% to about 8% (w/w), or from about 0.01% to about 7% (w/w), or from about 0.01% to about 6% (w/w), or from about 0.01% to about 5% (w/w), or from about 0.01% to about 4% (w/w), or from about 0.01% to about 3% (w/w), or from about 0.01% to about 2% (w/w), or from about 0.01% to about 1% (w/w), or from about 0.01% to about 0.75% (w/w), or from about 0.01% to about 0.5% (w/w), or from about 0.01% to about 0.25% (w/w), or from about 0.01% to about 0.10% (w/w), or from about 0.10% to about 10% (w/w), or from about 0.10% to about 9% (w/w), or from about 0.10% to about 8% (w/w), or from about 0.10% to about 7% (w/w), or from about 0.10% to about 6% (w/w), or from about 0.10% to about 5% (w/w), or from about 0.10% to about 4% (w/w), or from about 0.10% to about 3% (w/w), or from about 0.10% to about 2% (w/w), or from about 0.10% to about 1% (w/w), or from about 0.10% to about 0.75% (w/w), or from about 0.10% to about 0.5% (w/w), or from about 0.10% to about 0.25% (w/w), or from about 0.25% to about 10% (w/w), or from about 0.25% to about 9% (w/w), or from about 0.25% to about 8% (w/w), or from about 0.25% to about 7% (w/w), or from about 0.25% to about 6% (w/w), or from about 0.25% to about 5% (w/w), or from about 0.25% to about 4% (w/w), or from about 0.25% to about 3% (w/w), or from about 0.25% to about 2% (w/w), or from about 0.25% to about 1% (w/w), or from about 0.25% to about 0.75% (w/w), or from about 0.25% to about 0.5% (w/w), or from about 0.50% to about 10% (w/w), or from about 0.50% to about 9% (w/w), or from about 0.50% to about 8% (w/w), or from about 0.50% to about 7% (w/w), or from about 0.50% to about 6% (w/w), or from about 0.50% to about 5% (w/w), or from about 0.50% to about 4% (w/w), or from about 0.50% to about 3% (w/w), or from about 0.50% to about 2% (w/w), or from about 0.50% to about 1% (w/w), or from about 0.50% to about 0.75% (w/w), or from about 0.75% to about 10% (w/w), or from about 0.75% to about 9% (w/w), or from about 0.75% to about 8% (w/w), or from about 0.75% to about 7% (w/w), or from about 0.75% to about 6% (w/w), or from about 0.75% to about 5% (w/w), or from about 0.75% to about 4% (w/w), or from about 0.75% to about 3% (w/w), or from about 0.75% to about 2% (w/w), or from about 0.75% to about 1% (w/w), or from about 1% to about 10% (w/w), or from about 1% to about 9% (w/w), or from about 1% to about 8% (w/w), or from about 1% to about 7% (w/w), or from about 1% to about 6% (w/w), or from about 1% to about 5% (w/w), or from about 1% to about 4% (w/w), or from about 1% to about 3% (w/w), or from about 1% to about 2% (w/w), or from about 2% to about 10% (w/w), or from about 2% to about 9% (w/w), or from about 2% to about 8% (w/w), or from about 2% to about 7% (w/w), or from about 2% to about 6% (w/w), or from about 2% to about 5% (w/w), or from about 2% to about 4% (w/w), or from about 2% to about 3% (w/w), or from about 3% to about 10% (w/w), or from about 3% to about 9% (w/w), or from about 3% to about 8% (w/w), or from about 3% to about 7% (w/w), or from about 3% to about 6% (w/w), or from about 3% to about 5% (w/w), or from about 3% to about 4% (w/w), or from about 4% to about 10% (w/w), or from about 4% to about 9% (w/w), or from about 4% to about 8% (w/w), or from about 4% to about 7% (w/w), or from about 4% to about 6% (w/w), or from about 4% to about 5% (w/w), or from about 5% to about 10% (w/w), or from about 5% to about 9% (w/w), or from about 5% to about 8% (w/w), or from about 5% to about 7% (w/w), or from about 5% to about 6% (w/w), or from about 6% to about 10% (w/w), or from about 6% to about 9% (w/w), or from about 6% to about 8% (w/w), or from about 6% to about 7% (w/w), or from about 7% to about 10% (w/w), or from about 7% to about 9% (w/w), or from about 7% to about 8% (w/w), or from about 8% to about 10% (w/w), or from about 8% to about 9% (w/w), or from about 9% to about 10% (w/w) of antibacterial ingredients.

### Flavoring and Sweeteners

The composition of the present invention may contain a flavoring ingredient. The flavoring ingredient may be orange flavoring, apple flavoring, grapefruit flavoring, pineapple flavoring, strawberry flavoring, raspberry flavoring, cranberry flavoring, lime flavoring, lemon flavoring, grape flavoring, peach flavoring, any other fruit flavoring, vanilla flavoring, chocolate flavoring, caramel flavoring, mint flavoring, bubble gum flavoring, or any combination thereof.

Sweeteners such as aspartame, stevia, acesulfame, sucralose, maleic acid, citric acid, saccharin and the likes may also be included in the compositions of the present invention.

### Other Components

The composition of the present invention may contain other non-active excipients such as pigments and coloring agents, for example titanium oxide or other suitable pigments such as lactoflavins, chlorophylls such as copper derivatives of chlorophylls, and hydrogenated castor oil.

### Viscosity of the Composition

According to an embodiment, the viscosity of the oral care composition of the present invention may be from about 17500 to about 35000 cps, preferably 28800 cps, measured at 20° C. in a Brookfield apparatus at 20 rpm. The viscosity of the composition must be so that it does not prevent a good flow and good rinsing. The product is fully soluble in water.

### Storage Stability

The stability of the product was measured at 20° C. and 4° C. for 3 months. The product is placed in an oven and the physical and chemical characteristics measured that compare to the initial values. When he shows no phase separation, change in color, odor, or deposit, it is considered stable in storage for two years.

### Stability to Heat

The heat stability is carried out by the product in an oven at 45° C. for 45 days. it is verified that the physical and chemical parameters are identical to the initial values and the product has no phase change, color or odor. It is considered that the product is stable on storage in the heat.

### Density Measurement

Good density ensures gives a good texture to the product and influences it's holding in suspension and stability. The measured value is equal to 1.14 while the desired value is from 1.10 to 1.35.

Process for Preparation of Treated Calcium Carbonate (CaCO₃) Particles Having a Reduced or Inhibited Reaction with Fluoride

According to an embodiment, there is disclosed a method for the preparation of treated calcium carbonate (CaCO₃) particles having a reduced or inhibited reaction with fluoride from a fluoride compound suitable to provide beneficial fluoride treatment to teeth, the method comprising:
a) grinding an aragonite to obtain a coarse CaCO₃ powder;
b) sieving said coarse powder to obtain a first ground CaCO₃ powder having particle size of from about 60 microns to about 75 microns;
c) treating said first ground CaCO₃ powder with mildly acidic conditions at a pH of about 4.5 to 5.5, at a temperature sufficient and for a time sufficient to demineralize said first ground CaCO₃ powder and obtain a demineralized ground CaCO₃;
d) washing said demineralized ground CaCO₃ until a neutral pH is reached;
e) drying said demineralized ground CaCO₃, to obtain treated aragonite calcium carbonate (CaCO₃) particles having more than 95% (w/w) calcium carbonate content, and a specific surface area (SSA) of about 2.70 to about 3.1 m²/g.

In embodiments, the mildly acidic treatment may be performed with mild acids such as ammonium chloride, ammonium bromide, ammonium acetate, ammonium carbonate, ammonium phosphate, ammonium formate, ammonium malate, triammonium citrate, ammonium tartrate, acetic acid, citric acid, ascorbic acid, tannic acid, boric acids, lactic acid, formic acid, oxalic acid, uric acid, malic acid, tartaric acid, phosphorous acid and the likes. Stronger acids such as hydrochloric acid and phosphoric acids may also be used in dilute conditions that result in mildly acidic treatment of the calcium carbonate. The mild acid may be ammonium chloride or ammonium acetate, preferably ammonium chloride.

The concentration of ammonium chloride may be from about 0.1 M to about 10 M, preferably 1.87M (10% w/v). In the method of the present invention, step c) may be at a pH of about 4.5, of about 4.9, or at a pH of about 4.86.

In the method of the present invention, step c) may be at a temperature from about 65° C. to about 75° C.

In the method of the present invention, step d) may be performed in distilled water.

In the method of the present invention, step e) may be at about 200° C. to about 220° C.

In the method of the present invention, step e) may be at about 200° C.

In the method of the present invention, step e) may be for about 30 min to about 60 min., for example about 55 min.

According to an embodiment, the calcium carbonate is a calcite, an aragonite, a vaterite, or combinations thereof. Preferably, the calcium carbonate is an aragonite, and most preferably, the aragonite is oolittic aragonite.

The present invention will be more readily understood by referring to the following examples which are given to illustrate the invention rather than to limit its scope.

### Example 1

Preparation of an Abrasive Agent from Oolitic Aragonite

Oolitic aragonite samples comprise a chemical composition according to Table 1 below.

TABLE 1
chemical composition of oolitic aragonite

| | Percent by Weight | |
|---|---|---|
| Compound | Minimum | Maximum |
| CaCO₃ | 94 | 98.5 |
| MgCO₃ | 0.5 | 1.50 |
| SiO₂ | 0.02 | 0.08 |
| Fe₂O₃ | 0.008 | 0.025 |
| Al₂O₃ | 0.02 | 0.15 |
| SO₃ | 0.10 | 0.20 |
| NaCl | 0.06 | 0.25 |
| SrO | 0.30 | 1.25 |

The oolittic aragonite samples are provided as chips and powdered material having a particle size distribution according to Table 2 below.

TABLE 2
particle size distribution

| Mesh opening | U.S. Standard | Percent |
|---|---|---|
| in micron | Mesh number | retained |
| 75 | 200 | 99-100 |
| 106 | 140 | 93-99 |
| 150 | 100 | 80-98 |
| 300 | 50 | 40-67 |
| 600 | 30 | 10-26 |
| 1180 | 16 | 3-11 |
| 2360 | 8 | 1-4 |
| 4750 | 4 | 0-1 |

Table 2 shows that almost all chips and powdered material from the aragonite sample are comprised of particles having a particle size 75 μm or greater, as 99-100% are retained upon a 200-mesh size screen.

The raw material oolittic aragonite is stored in a hopper equipped with a screw feeder and subsequently ground in an ultra-centrifugal type rotor mill and sieved with a sieve having a cut-off between 55 and 65 μm. On the one end, the coarse portion (65%) coming out of the sifter is returned to the feed hopper of the mill to be ground further. On the other end, the fine portion (representing the raw material for the formulation of the abrasive agent) is stored temporarily in a hopper.

This oolittic aragonite powder is then transferred to a double-walled impervious sealed reactor with a condenser and a vent. Firstly, water preheated with steam is mixed with the oolittic aragonite powder using a solid-liquid premix pump. This step is to prevent the generation of dust in the reactor and clogging of the condenser as well as the loss of powder during changing of the reactor. Secondly, ammonium chloride is added to the reactor and viscosity of the clay is measured at 20° C. to be between 400 and 800 centipoises. The mass proportions were measured to be at 55% water, 35% aragonite powder and 10% ammonium chloride (1 M). Once this formulation is complete, the mixture is heated to a temperature between 8° and 90° C., reacted with agitation to equilibrium (about 3 to 4 hours), and left to cool without agitation for about 2 to 3 h. The reaction mixture is filtered in a basket centrifuge with porosity of 55 μm and washed with water until a neutral pH is obtained. Finally, the mix is dried in a tunnel dryer and the treated oolittic aragonite powder is collected at the dryer outlet. A suitable preservative agent is added and homogenized with the powder in a double-cone mixer before being poured into a hopper and packaged in plastic bags.

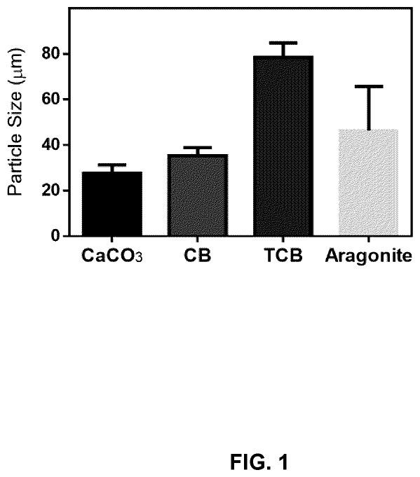

Particle size of the cuttlefish bone powder (CB): treated cuttlefish bone powder (TCB), synthetic calcium carbonate (CaCO₃), and treated Oolitic aragonite powder was analyzed using a Microtrac Dynamic Image Analysis (DIA) particle size analyzer. The sample of treated Oolitic aragonite was dispersed in isopropanol. Ultrasonicated for 1 min and measured. Oolitic Referring to FIG. 1 and Table 3, CB is shown to have particle size of about 35.371±3.472 μm, TCB of about 78.422±6.441 μm, synthetic calcium carbonate (CaCO₃) of about 27.550±3.781 μm and treated oolitic aragonite of about 46.38±19.36 μm.

TABLE 3
Particle size

| Sample | Particle Size |
|---|---|
| CB | 35.371 ± 3.472 |
| TCB | 78.422 ± 6.441 |
| CaCO₃ | 27.550 ± 3.781 |
| Treated oolitic aragonite | 46.38 ± 19.36 |

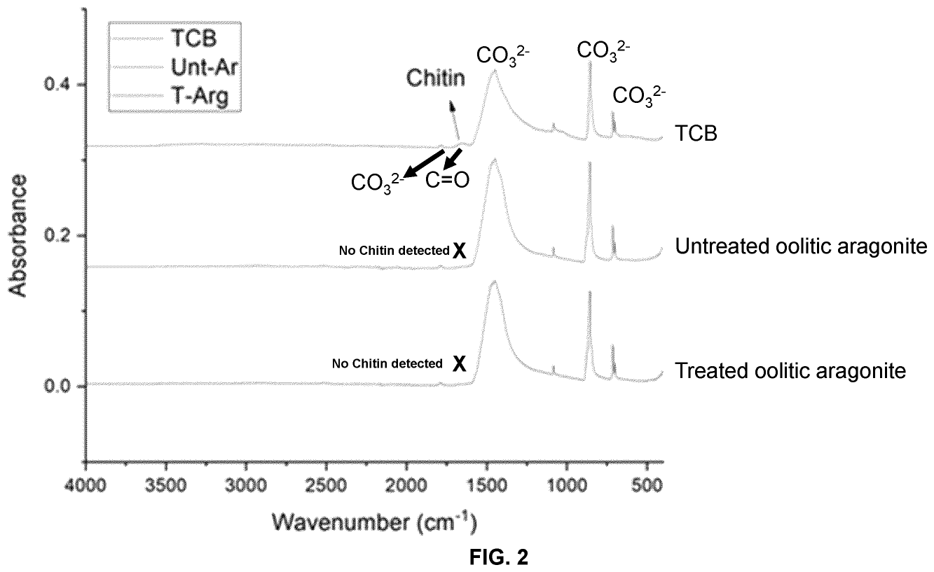

Next, the TCB, the untreated oolitic aragonite, and the treated Oolitic aragonite were subjected to Fourier-transform infrared spectroscopy (FTIR) analysis. Now referring to FIG. 2, which shows the FTIR spectra of treated oolitic aragonite compared to a treated cuttlefish bone aragonite (TCB) and untreated oolitic aragonite samples. The peaks associated with the CO₃²⁻ ion are readily identifiable in each sample. However, the C═O peak identified in the TCB, which is associated with the presence of chitin in the TCB sample, is absent from the treated Oolitic aragonite. Indeed, the treated Oolitic aragonite Spectrum showed the absence of the C═O band at 1653 cm⁻¹ corresponding to Chitin.

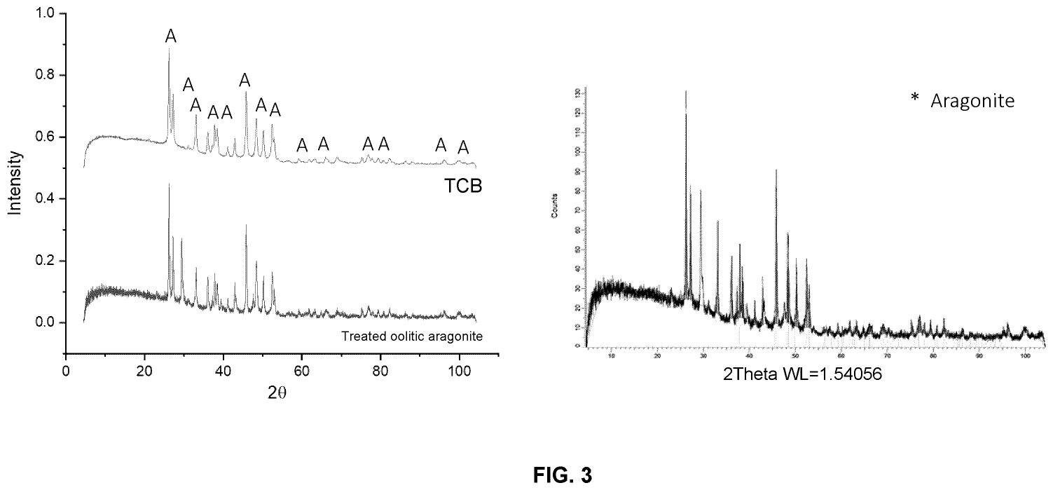

Now referring to FIG. 3, it is shown a comparison of treated Oolitic aragonite (bottom left) and TCB (top right). The diffractogram shows that the TCB shows wider band width, which indicates smaller particle size.

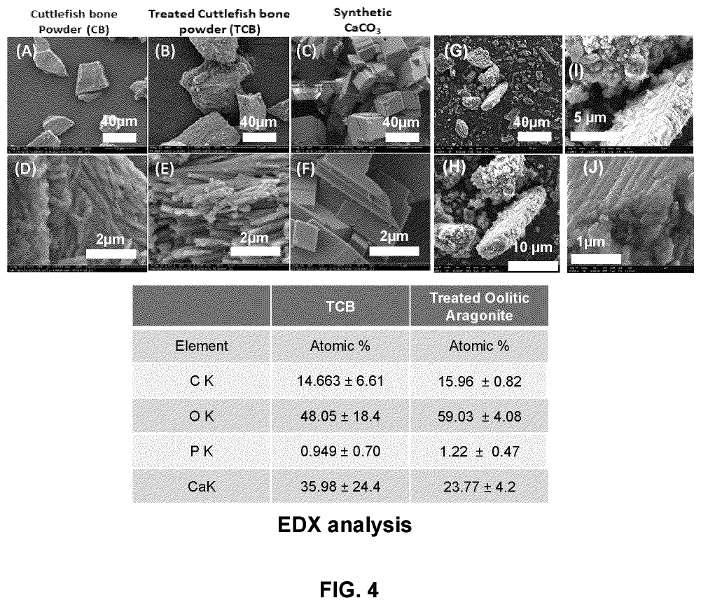

Now referring to FIGS. 4A to J, there is shown SEM of CB (FIGS. 4A and 4D), TCB (FIGS. 4B and 4D), synthetic calcium carbonate (FIGS. 4C and 4F) and treated Oolitic aragonite (FIG. 4G, 4H, 4I or 4J). The SEM micrographs show that CB (FIGS. 4A and 4D) has a needle-like structure, contrasting to the cuboidal structure of CaCO₃ (FIGS. 4C and 4F). Treating CB to produce TCB increased surface texturing as the needle-like structures became more defined and separated. Treating Oolitic aragonite to produce treated Oolitic aragonite also increased surface texturing, and the surface displayed an even lumpier and clumpier surface than TCB. Now referring to FIG. 4K, the EDX analysis shows the elemental composition of both TCB and oolitic aragonite are mainly of calcium, carbon and oxygen, which is compatible with calcium carbonate aragonite and traces of phosphorus, although oolitic aragonite also has some traces of calcium phosphate. The presence of calcium and phosphorus could be beneficial for the tooth.

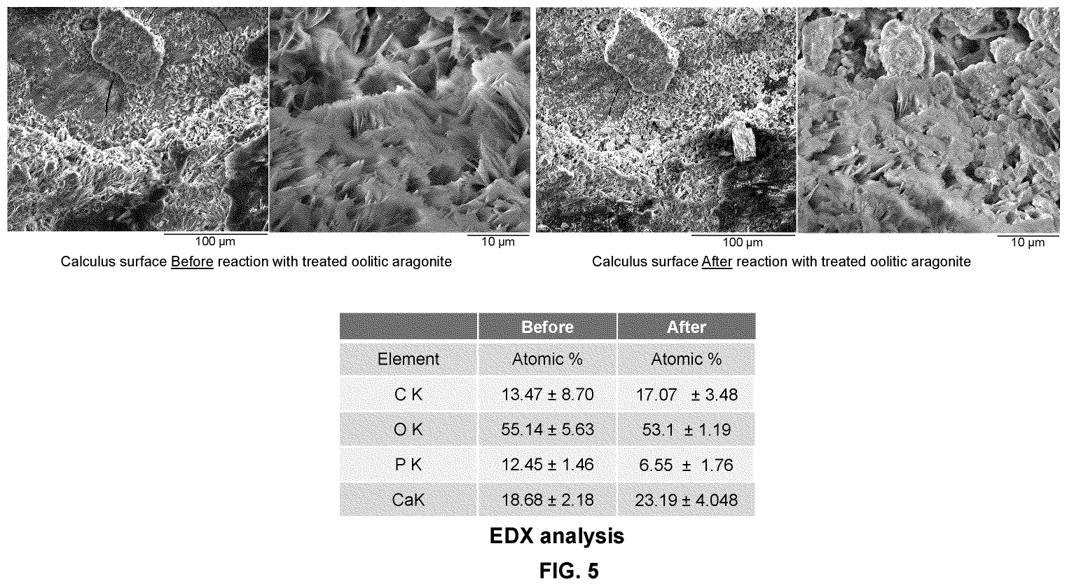

Now referring to FIG. 5, the top panels are SEM of calculus before reaction with treated oolitic aragonite (left), and after treatment with treated oolitic aragonite (right), at two distinct magnifications. The addition of treated oolitic aragonite results in a change of appearance that suggests that a layer of treated oolitic aragonite has deposited on the calculus. FIG. 5 bottom is an EDX analysis that shows a change in the chemical composition of calculus, particularly the P-K which almost doubled and indicative of a loss of phosphate from the calculus, and the CaK which may have increased slightly. In FIG. 5, the elemental analysis of dental calculus before and after exposure to oolitic aragonite reveals a substantial decrease in phosphate content at the expense of an increase in calcium and carbon. This seems to indicate changes in the surface composition of the dental calculus favoring the formation of calcium carbonate species at the expense of calcium phosphate species. The increase of calcium and the loss of phosphorus content confirm the reaction of calculus with the treated oolitic aragonite.

Next, the BET specific surface area (SSA) were determined from a five-point nitrogen adsorption isotherm at 77.3 K (Micromeritics Tristar 3000) from the particles collected on the filter. About 0.5 gram of each sample were degassed in nitrogen at 120° C. overnight (Micromeritics Flow prep 060) before adsorption to remove water bound to their surface from air moisture.

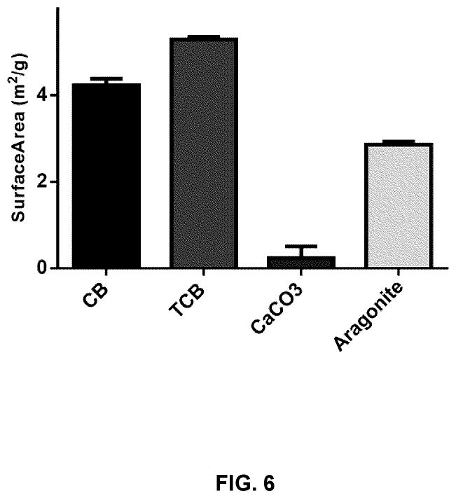

Referring to FIG. 6A and Table 4, CB is shown to have a SSA of about 4.23±0.15 m²/g, TCB a SSA of about 5.29±0.06 m²/g, synthetic calcium carbonate (CaCO₃) a SSA of about 0.2380±0.27 and treated oolitic aragonite a SSA of about 2.8574±0.07.

TABLE 4
Specific Surface Area (SSA) of samples

| Sample | Surface Area |
|---|---|
| CB | 4.2300 ± 0.15 |
| TCB | 5.2900 ± 0.06 |
| CaCO₃ | 0.2380 ± 0.27 |
| Treated oolitic aragonite | 2.9036 ± 0.1198 |

Next, the crystalline nature of triplicate samples of CB, TCB, synthetic calcite powder (CaCO₃), oolitic aragonite and treated oolitic aragonite were studied using X-ray diffraction (XRD), (Bruker AXS GmbH, Karlsruhe, Germany diffractometer), using a Cu Kα radiation source (λ Kα=1.5406 Å), operated at 40 kV and 40 mA, within the 10 to 60° range in 20 in a step scan mode with steps of 0.02° and counting time of 4 seconds per step. As shown in Table 5, the crystallinity (in %) of CB was measured to be 22.466±1.050, TCB of 36.4±0.692, calcite CaCO₃ of 44.166±0.513, oolitic aragonite (untreated) of 34.033±0.680 and treated oolitic aragonite of 26.133±2.223.

TABLE 5
Crystallinity of samples

| Sample | Sample 1 | Sample 2 | Sample 3 | Crys.% |
|---|---|---|---|---|
| CB | 23.5 | 22.5 | 21.4 | 22.466 ± 1.050 |
| TCB | 37.2 | 36 | 36 | 36.4 ± 0.692 |
| CaCO₃ | 44.6 | 44.3 | 43.6 | 44.166 ± 0.513 |
| Arg | 34.8 | 33.8 | 33.5 | 34.033 ± 0.680 |
| T-Arg | 24.9 | 24.8 | 28.7 | 26.133 ± 2.223 |

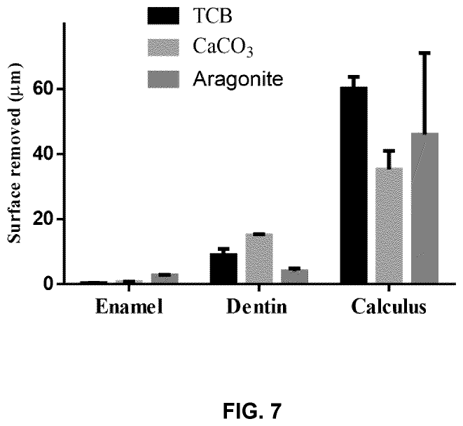

Now referring to FIG. 7. Next, a brushing test was performed for each of TCB, synthetic calcium carbonate (CaCO₃) and treated oolitic aragonite. Abrasiveness of the TCB and synthetic calcite powder (i.e., the synthetic calcium carbonate) was assessed using a brushing test where polished resin-embedded Enamel/Dentin/calculus sections were mounted as described in WO2021062554 and attached to a customized brushing machine (Mach-1, Biomomentum, QC) in a specifically designed mold that only exposed 0.5 mm×15 mm of the sample to the brush. Customized toothbrush was fixed in the machine parallel to the sample surface and slurries of TCB, and synthetic calcite powder and treated oolitic aragonite in dd-H₂O 1:1 (w:w) were used to brush the calculus, dentin, and enamel sections in the machine for 56 minutes at 90 strokes/min, (5400 cycles), under a load of 500 g. This is equivalent to regular tooth brushing of 2-minute per session, twice a day for 2 weeks. The effect of the slurries on the calculus removal was determined by measuring the abrasion depth using a stylus profilometer. The abrasion depth was measured using a stylus profilometer (Dektak XT™, Bruker, United States) using the sample surface that was not in touch with the brush as the baseline. The deepest point in each sample profile was registered and compared to the baseline. In FIG. 7, the samples are, from left to right, TCB, synthetic calcium carbonate and treated oolitic aragonite for each surface tested. The results show that all 3 slurries have low abrasivity for enamel despite treated oolitic aragonite being the most abrasive of the 3. Unexpectedly, treated oolitic aragonite shows low abrasivity against dentin, which is a desired feature, especially considering that it displays comparable abrasivity as TCB against calculus.

### Example 2

Reaction with Calculus

To investigate mineral content of calculus, samples of calculus were collected, and they were analyzed in a synchrotron facility by comparing them to pure standards of calcium acetate (CaAc), amorphous calcium phosphate (ACP), α-tricalcium phosphate (α-TCP), β-tricalcium phosphate (β-TCP), dicalcium phosphate dihydrate (DCPD, in the form of brushite), octacalcium phosphate (OCP), hydroxyapatite (HA), crystalline hydroxyapatite (CHA), and aragonite (CaCO₃).

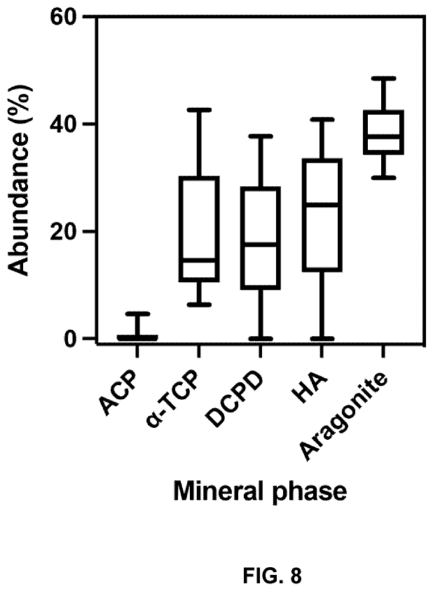

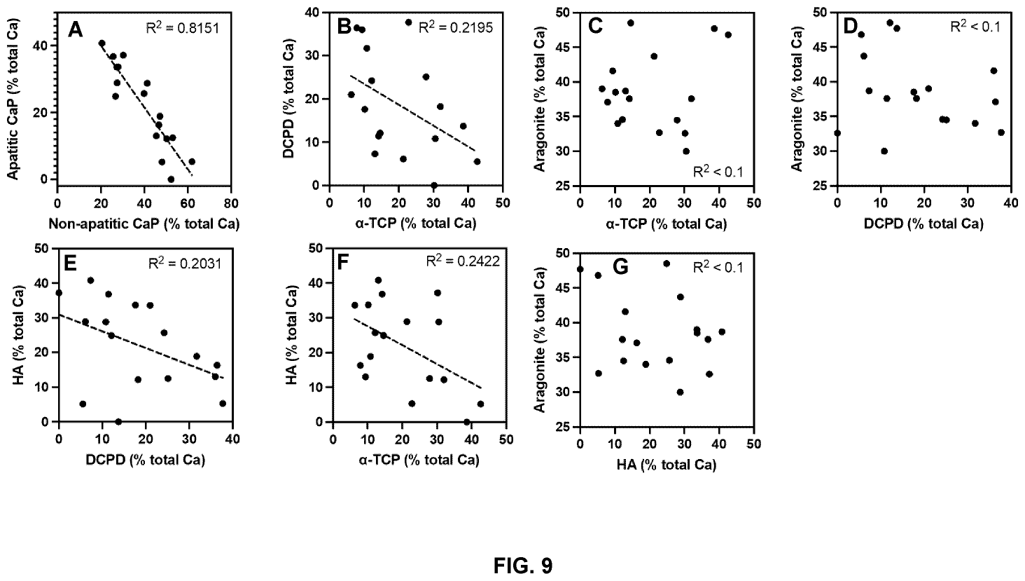

Now referring to FIG. 8, which shows the mineral phases found in the calculus samples. A small amount of amorphous calcium phosphate was identified, as well as appreciable quantities of α-TCP, DCPD, HA and aragonite. Next, the amounts measured for each of non-Apatitic calcium phosphate in the calculus samples were plotted against the Apatitic calcium phosphate [i.e. the total of hydroxyapatite (HA) and crystalline hydroxyapatite (CHA)](FIG. 9A), α-TCP vs. DCPD (FIG. 9B), α-TCP vs. Aragonite (FIG. 9C), DCPD vs. Aragonite (FIG. 9D), DCPD vs. HA (FIG. 9E), α-TCP vs. HA (FIG. 9F), HA vs. Aragonite (FIG. 9G). A very strong correlation was identified between non-Apatitic calcium phosphate and Apatitic calcium phosphate, suggesting that there may be a link between the abundance of these two calcium phosphate phases, and possibly a maturation mechanism between the two. None of the other comparisons between calcium phosphate phases and calcium phosphates yielded meaningful correlations, suggesting that they might form independently.

Next, to investigate the reaction between the compounds identified in dental calculus and calcite CaCO₃, TCB, or treated oolitic aragonite, equal molar amounts of the compounds were mixed and incubated in H₂O or not, and the samples were subjected to Fourier-transform infrared spectroscopy (FTIR) analysis. To identify the functional groups, Infrared (IR) spectra of the powder samples were acquired using a Bruker Tensor 27 Fourier transform infrared (FTIR) spectrometer with accumulation of 64 scans in the range of 400-4000 cm⁻¹ at a resolution of 4 cm⁻¹.

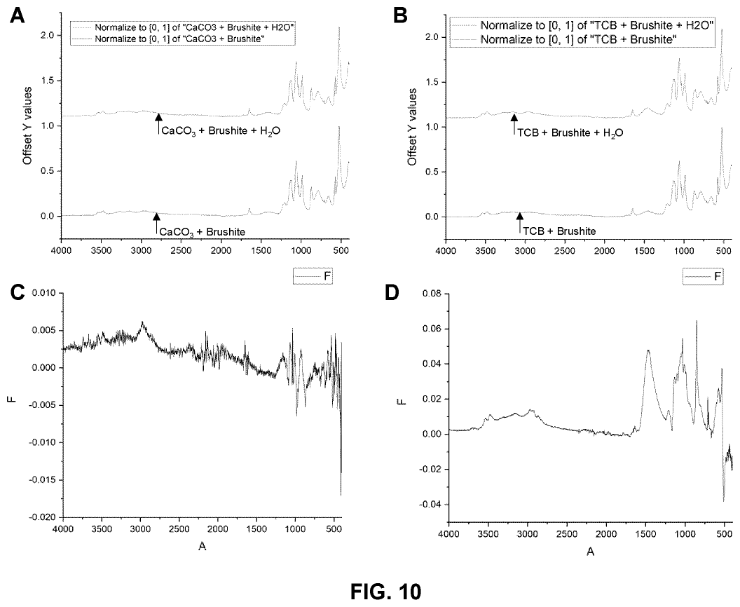

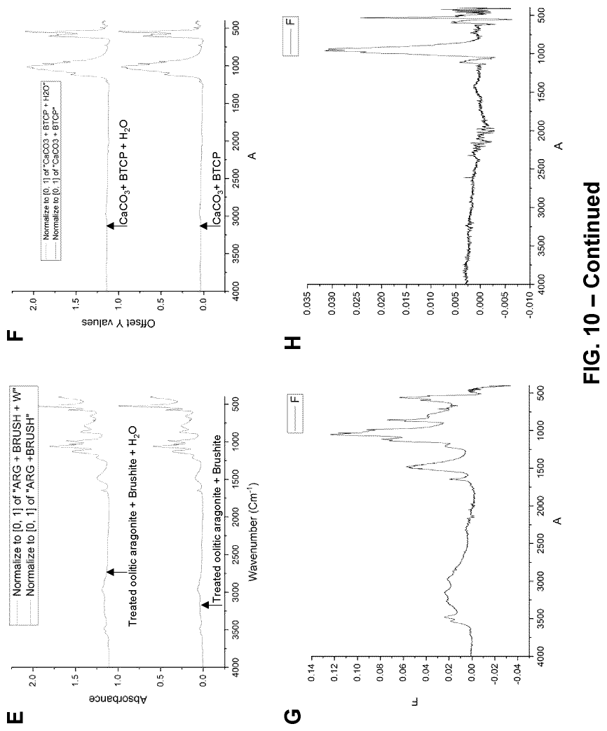

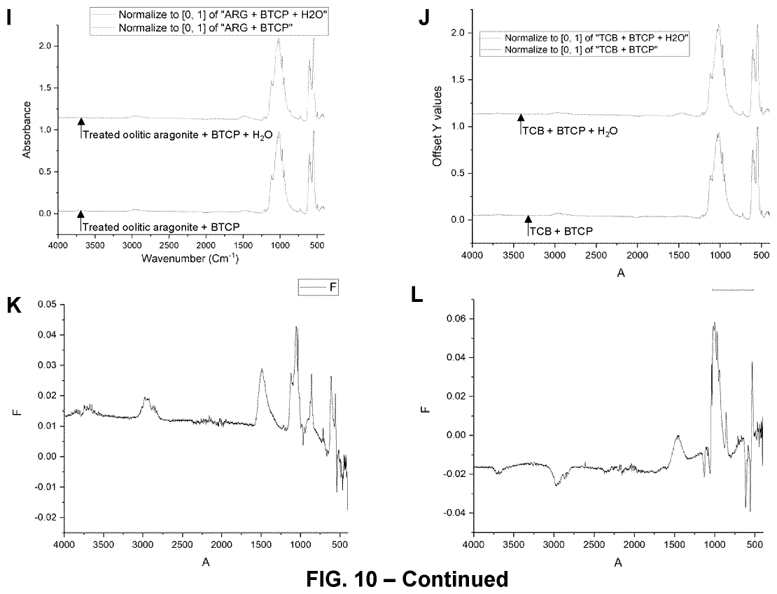

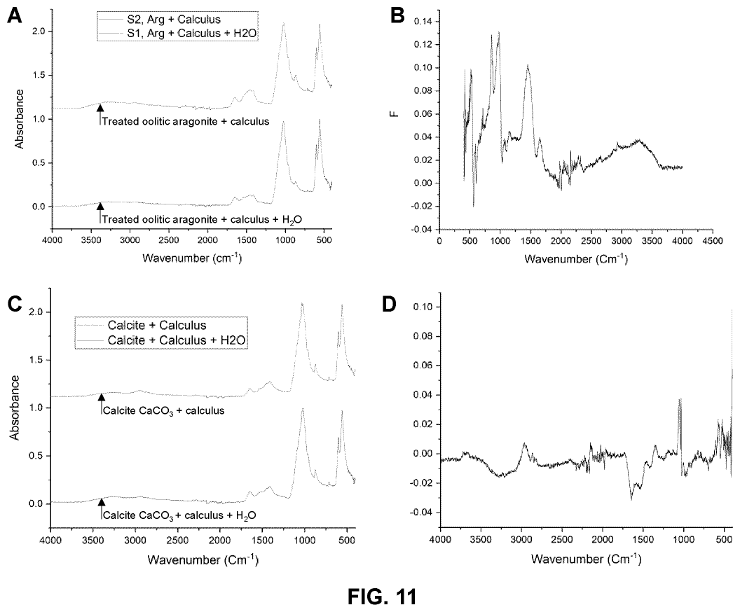

Now referring to FIGS. 10A to 10L. FIGS. 10A and 10C shows that there is no reaction between calcite CaCO₃ and brushite DCPD. FIGS. 10B and 10D shows that there is a reaction taking place between TCB and brushite DCPD. Likewise, FIGS. 10E and 10G shows that there is a reaction taking place between treated oolitic aragonite and brushite DCPD. FIGS. 10F and 10H shows that there is only a weak reaction between calcite CaCO₃ and β-tricalcium phosphate (BTCP). Likewise, FIGS. 101 and 10K shows that there is only a weak reaction taking place between treated oolitic aragonite and BTCP. FIGS. 10J and 10L shows that there is only a weak reaction taking place between TCB and BTCP. This experiment help explains the mechanism by which oolitic aragonite reacts with dental calculus. The results shown in FIG. 10 reveal that calcium carbonates are able to react with calcium phosphate species found in dental calculus such as brushite and tri calcium phosphate. And among the 2 calcium carbonates tested (calcite and aragonite) the oolitic aragonite is the most reactive one.

Next, to investigate the reaction between dental calculus and calcite CaCO₃, or treated oolitic aragonite, equal molar amounts of the compounds were mixed and incubated in H₂O or not, and the samples were subjected to Fourier-transform infrared spectroscopy (FTIR) analysis. Now referring to FIGS. 11A to 11D. FIGS. 11A and 11B shows that there is a reaction taking place between treated oolitic aragonite and calculus. FIGS. 11C and 11D shows that there is no or very little reaction taking place between calcite CaCO₃ and calculus. Overall, FIG. 11 demonstrates how oolitic aragonite reacts with dental calculus. This figure shows the reactivity dental calculus with calcium carbonate. The figures show the FTIR spectra of a mixture of dental calculus powder with calcium carbonate powder before and after exposure to water. The results show that after exposure to water, there is a change in the chemical composition of the powder mixtures, and this change is more pronounced in the oolitic aragonite/dental calculus mixture.

### Example 3

Reactivity of Aragonite with Free Ca²⁺ and PO₄³⁻

The impact of calcium carbonate powders including aragonite, treated aragonite, and calcite on the precipitation of calcium phosphate [Ca₃(PO₄)₂], was investigated. In a brief, 15.0 mM CaCl₂) and 15.0 mM Na₂HPO₄ were added to prepare calcium phosphate supersaturated solutions in a covered glass beaker. The pH of the solution was adjusted to 5.60±0.2. After adding 0.5 grams of aragonite, treated aragonite, or synthetic calcite (CaCO₃) powder, 1.0 M hydrochloric acid (HCl) (Sigma-Aldrich, USA) was added gradually at 20° C. The natants were collected after 1 hour, or 1, 3, 7, 14, and 21 days by centrifuging the precipitate for 15 minutes at 10,000 rpm. Following drying, XRD and FTIR analyses of these natants were conducted. In a parallel experiment, calcium and phosphate ion concentrations were measured after 14 days using inductively coupled plasma atomic emission spectroscopy (ICP-AES; Thermo Scientific iCAP 6500 dual view, UK). As a control solution, pure calcium phosphate solution was prepared and stored under the same conditions.

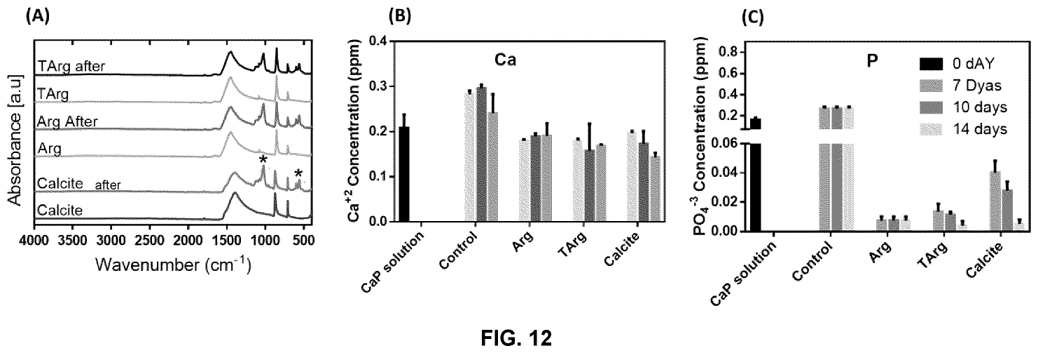

FIG. 12 depicts calcium phosphate precipitation in the presence of aragonite, treated aragonite, or synthetic calcite (FIG. 12A). After 14 days of incubation in a supersaturated solution of calcium phosphate, the FTIR spectra of aragonite, treated aragonite, or synthetic calcite changed, showing signals for the PO₄³⁻ bands at 1035, 1023, 600, and 560 cm⁻¹ (FIG. 12A). This implies that the calcium carbonate can react with free PO₄³⁻ ions and remove them from the surrounding solution.

The variation in ion concentrations (Ca²⁺ and PO₄³⁻ ions) in the solution changed after exposure to the aragonite, treated aragonite, or synthetic calcite powder (FIGS. 12B and 12C). After 14 days of immersion, the concentration of Ca²⁺ in the calcium phosphate solution slightly decreased (FIG. 12B), while the concentration of PO₄³⁻ ions decreased substantially (FIG. 12C), upon exposure to aragonite, treated aragonite, or synthetic calcite. The results also indicate that the speed of reaction was faster with aragonite and treated aragonite than with calcite. With calcite, the concentration decreased between day 7 and 14 while with the aragonite and treated aragonite, no change occurred between day 7 and day 14 days indicating that the reaction reached equilibrium earlier. It was found that the concentration of PO₄³⁻ ions is lower in the aragonite and treated aragonite samples compared to synthetic calcite, indicating that more PO43− ions have precipitated on the aragonite and treated aragonite than on calcite. These results suggest that the aragonite and treated aragonite act as scavenger for free phosphate. In fact, these results suggest that aragonite and treated aragonite can react with free Ca²⁺ and PO₄³⁻ ions and remove them from the aqueous environment. This suggest that aragonite could act as inhibitor of mineralization and calculus formation. Previous work on the management of dental calculus has focused on calcium chelation, such as the use of pyrophosphate and carboxylate compounds. These molecules prevent deposition of calculus, but they are not able to remove calculus. Moreover, if used at high concentration, they can cause the unwanted demineralization of the tooth. The results presented here show an unexpected new strategy to manage dental calculus that focuses on phosphate removal instead of calcium chelation, without damaging the tooth.

### Example 4

Formulations with Treated Oolitic Aragonite

Formulations comprising TCB as the abrasive were prepared according to the following recipes:

TABLE 6
TCB based formulation

| | | | | | +0.25% |
|---|---|---|---|---|---|
| | | | | | bicarb & |
| | Control | Control | +0.5% | +0.5% | 0.25% |
| Ingredients (% w/w) | 1 | 2 | bicarb | TSPP | TSPP |
| Water | 28.9 | 28.9 | 28.4 | 28.4 | 28.4 |
| Sorbitol | 20.7 | 20.7 | 20.7 | 20.7 | 20.7 |
| TCB aragonite | 11.5 | 11.5 | 11.5 | 11.5 | 11.5 |
| abrasive | | | | | |
| Zeodent 165 | 11.5 | 0.0 | 0.0 | 0.0 | 0.0 |
| (Thickener) | | | | | |
| Sylodent SM990T | 0.0 | 11.5 | 11.5 | 11.5 | 11.5 |
| (Thickener) | | | | | |
| Glycerol | 10.0 | 10.0 | 10.0 | 10.0 | 10.0 |
| Xylitol | 10.0 | 10.0 | 10.0 | 10.0 | 10.0 |
| Sodium Citrate | 2.0 | 2.0 | 2.0 | 2.0 | 2.0 |
| Flavor | 1.22 | 1.22 | 1.22 | 1.22 | 1.22 |
| Xanthan gum | 0.5 | 0.5 | 0.5 | 0.5 | 0.5 |
| (KELDENT L# | | | | | |
| 9H8041K) | | | | | |
| Na Lauryl | 0.9 | 0.9 | 0.9 | 0.9 | 0.9 |
| Sarcosinate | | | | | |
| TiO₂ | 0.5 | 0.5 | 0.5 | 0.5 | 0.5 |
| Na Saccharin | 0.3 | 0.3 | 0.3 | 0.3 | 0.3 |
| Cellulose gum | 0.5 | 0.5 | 0.5 | 0.5 | 0.5 |
| (Aqualon CMC | | | | | |
| 12M31P) | | | | | |
| Na benzoate | 0.16 | 0.16 | 0.16 | 0.16 | 0.16 |
| Na bicarbonate | 0.1 | 0.1 | 0.6 | 0.1 | 0.35 |
| TSPP | 0.0 | 0.0 | 0.0 | 0.5 | 0.25 |
| K sorbate | 0.08 | 0.08 | 0.08 | 0.08 | 0.08 |
| Menthol | 0.06 | 0.06 | 0.06 | 0.06 | 0.06 |
| Spearmint leaf oil | 0.01 | 0.01 | 0.01 | 0.01 | 0.01 |
| Sodium MFP | 1.14 | 1.14 | 1.14 | 1.14 | 1.14 |
| Total | 100.0 | 100.0 | 100.0 | 100.0 | 100.0 |

The purpose of testing was to determine the total fluorine (fresh) and total soluble available fluorine (fresh and aged) of five sodium monofluorophosphate dentifrices. For this purpose, the samples were incubated 90 days at 40°±2° C. and 75%±5% humidity, to simulate normal aging of products at room temperature for a period of two years. The tests performed are as follows:

### Total Fluoride Test

The test was performed in triplicate on fresh product. The insides of Petri dish covers were coated with sodium hydroxide by placing 0.3 ml of 0.25 N ethanolic sodium hydroxide and allowing the alcohol to evaporate under vacuum. Samples of 0.25 g of each dentifrice (weighed to 0.001 g) were mixed with 25.0 ml of de-ionized water using a non-aerating mixer (1:100). 2.0 ml of each slurry was transferred to the bottom of a Petri dish. To each Petri dish, 4.0 ml of 70% HClO₄ was added. A sodium hydroxide-covered lid was immediately placed over each slurry in the bottom half of a Petri dish. Each dish was then placed in an oven at 60° C. overnight.

After removing the Petri dishes from the oven, the lids were immediately removed. The lids were washed multiple times with a few ml of de-ionized water. The wash water was collected in a 25 ml volumetric flask. Each flask was then brought to volume with de-ionized water. After diluting 1:1 with TISAB II, this solution was analyzed for fluoride using a fluoride ion specific electrode and a pH/ISE meter. A standard fluoride curve was similarly prepared (hydrolyzed and diluted) and used for determination of the fluoride content of each of the dentifrice slurries.

### Test for Total Soluble Available Fluorine

This test was performed in triplicate on fresh product. The insides of Petri dish covers was coated with sodium hydroxide by placing 0.3 ml of 0.25 N ethanolic sodium hydroxide and allowing the alcohol to evaporate under vacuum. Samples of 2.5 g of each dentifrice (weighed to 0.001 g) were mixed with 25.0 ml of de-ionized water using a non-aerating mixer (1:10 dilution). Each slurry was then centrifuged at 11,000 g for 8 minutes. 2.0 ml of each supernatant solution was transferred to the bottom of a Petri dish. To each Petri dish, 4.0 ml of 70% HClO₄ was added. A sodium hydroxide-covered lid was immediately placed over each slurry in the bottom half of a Petri dishes. The dishes were then placed in an oven at 60° C. overnight.

After removing the Petri dishes from the oven, the lids were immediately removed. Each lid was washed multiple times with a few ml of de-ionized water. The wash water was collected in 25 ml volumetric flasks. Each flask was then brought to volume with de-ionized water. After diluting 1:1 with TISAB II, these solutions were analyzed for fluoride using a fluoride ion specific electrode and a pH/ISE meter. A standard fluoride curve was similarly prepared (hydrolyzed and diluted) and used for determination of the fluoride content of the slurries.

### Aging

The test products were aged for 90 days in 40°±2° and 75%±5% humidity and is analyzed again for total soluble available fluorine as described above.

TABLE 7
Bioavailability of Fluoride after aging

| | Fluoride (ppm) | | |
|---|---|---|---|
| | | | Aged |
| Toothpaste | Total | Bioavailable | bioavailable |
| Control 1 | 1521 | 1521 | 800 |
| Control 2 | 1491 | 1513 | 807 |
| +0.5% bicarb | 1531 | 1458 | 834 |
| +0.5% TSPP | 1518 | 1448 | 599 |
| +0.25% bicarb & | 1508 | 1499 | 791 |
| 0.25% TSPP | | | |

The results show that dentifrice formulations containing TCB aragonite abrasives display a loss of bioavailable fluoride of about 50% (52% and 53% remaining in the two controls). The addition of an increased concentration of sodium bicarbonate (+0.5%) only slightly increase the bioavailability of fluoride (about 57%). Addition of tretrasodium pyrophosphate (TSPP) decreased the bioavailability even more that in the control, to about 41%. Use of a mixture of +0.25% bicarbonate and 0.25% TSPP provided bioavailability of 52%, comparable to the control dentifrice compositions. Control 1 and 2 differ in their composition in the thickener/abrasive hydrated SiO₂ used. Both the Zeodent 165™ and Sylodent SM990T performed equally well.

Next, in view of the performance of TCB described above, new formulations comprising treated oolitic aragonite as the abrasive were prepared for comparison to TCB formulations, according to the following recipes:

TABLE 8
TCB and treated oolitic aragonite-based formulation

| | | | +0.5% | +0.5% |
|---|---|---|---|---|
| | | | bicarb | bicarb |
| Ingredients (% w/w) | Control-1 | Control-2 | V1 | V-2 |
| Water | 28.9 | 28.9 | 28.4 | 28.4 |
| Sorbitol | 21.7 | 21.7 | 21.7 | 21.7 |
| TCB aragonite | 11.5 | 0.0 | 11.5 | 0.0 |
| CaCO₃ (Abrasive) | | | | |
| Treated oolitic | 0.0 | 11.5 | 0.0 | 11.5 |
| aragonite CaCO₃ | | | | |
| (Abrasive) | | | | |
| Sylodent SM990T | 10.5 | 10.5 | 10.5 | 10.5 |
| (Thickener) | | | | |
| Glycerol | 10.0 | 10.0 | 10.0 | 10.0 |
| Xylitol | 10.0 | 10.0 | 10.0 | 10.0 |
| Sodium Citrate | 2.0 | 2.0 | 2.0 | 2.0 |
| Flavor | 1.22 | 1.22 | 1.22 | 1.22 |
| Xanthan gum | 0.5 | 0.5 | 0.5 | 0.5 |
| (KELDENT L# | | | | |
| 9H8041K) | | | | |
| Na Lauryl Sarcosinate | 0.9 | 0.9 | 0.9 | 0.9 |
| TiO₂ | 0.5 | 0.5 | 0.5 | 0.5 |
| Na Saccharin | 0.3 | 0.3 | 0.3 | 0.3 |
| Cellulose gum | 0.5 | 0.5 | 0.5 | 0.5 |
| (Aqualon CMC | | | | |
| 12M31P) | | | | |
| Na benzoate | 0.16 | 0.16 | 0.16 | 0.16 |
| Na bicarb | 0.1 | 0.1 | 0.6 | 0.6 |
| TSPP | 0.0 | 0.0 | 0.0 | 0.0 |
| K sorbate | 0.08 | 0.08 | 0.08 | 0.08 |
| Menthol | 0.06 | 0.06 | 0.06 | 0.06 |
| Spearmint leaf oil | 0.01 | 0.01 | 0.01 | 0.01 |
| Sodium MFP | 1.14 | 1.14 | 1.14 | 1.14 |
| Total | 100.0 | 100.0 | 100.0 | 100.0 |

The purpose of testing was to determine the total fluorine (fresh) and total soluble available fluorine (fresh and aged) of these sodium monofluorophosphate dentifrices. For this purpose, the samples were incubated 90 days at 40°±2° C. and 75%±5% humidity, to simulate normal aging of products at room temperature for a period of two years. The tests performed are as detailed above.

TABLE 9
Bioavailability of Fluoride after aging

| | Fluoride (ppm) | | |
|---|---|---|---|
| Toothpaste | Total | Bioavailable | Aged bioavailable |
| TCB | 1558 | 1526 | 896 |
| Treated oolitic | 1681 | 1621 | 1317 |
| aragonite | | | |
| TCB + 0.5% | 1608 | 1442 | 1038 |
| bicarbonate | | | |
| Treated oolitic | 1667 | 1495 | 1454 |
| aragonite + 0.5% | | | |
| bicarbonate | | | |

The results show that dentifrice formulations containing TCB aragonite abrasives display a loss of bioavailable fluoride of more than 40% (59% remaining in the TCB sample). The addition of an increased concentration of sodium bicarbonate (+0.5%) increases the bioavailability of fluoride (about 72%). Unexpectedly, in the dentifrice formulations containing treated oolitic aragonite abrasives, the loss of bioavailable fluoride is much less, with only a loss of 19% (81% remaining in the treated oolitic aragonite sample), a two-fold improvement over the TCB-containing dentifrice. Surprisingly, the addition of an increased concentration of sodium bicarbonate (+0.5%) increases the bioavailability of fluoride (about 97%); which is close to no loss in bioavailable fluoride.

Next, the aging properties of these 4 dentifrices were evaluated, particularly the viscosity and pH of the samples over a period of up to 68 days. The viscosity is an important parameter to monitor to estimate the visual and aesthetical appearance of the product as it ages. The pH is an important parameter to monitor to avoid elevated pH values that are compatible with increased tartar and calculus formation.

TABLE 10
Viscosity and pH of dentifrice over time

| | Initial | | | | 1-days | | | |
|---|---|---|---|---|---|---|---|---|
| | Viscosity, cps, 1 min | | | | Viscosity, cps, 1 min | | | |
| | TE/5 rpm | | | pH | TE/5 rpm | | | pH |
| | Jul. 21, 2021 | TE/10 rpm | TF/20 rpm | 10% | Jul. 22, 2021 | TE/10 rpm | TF/20 rpm | 10% |
| TCB | 189 000 | 115 000 | 81 500 | 8.07 | 234 000 | 146 000 | 93 000 | 8.21 |
| Treated oolitic | 144 000 | 95 500 | 66 000 | 7.55 | 176 000 | 113 000 | 76 000 | 7.74 |
| aragonite | | | | | | | | |
| TCB + 0.5% | 197 000 | 130 000 | 87 000 | 8.26 | 236 000 | 152 000 | 100 000 | 8.40 |
| bicarbonate | | | | | | | | |
| Treated oolitic | 169 000 | 112 000 | 76 000 | 8.15 | 202 000 | 127 000 | 87 500 | 8.32 |
| aragonite + 0.5% | | | | | | | | |
| bicarbonate | | | | | | | | |

| 7-days | | | | 21-days | | | |
|---|---|---|---|---|---|---|---|
| Viscosity, cps, 1 min | | | | Viscosity, cps, 1 min | | | |
| TE/5 rpm | | | pH | TE/5 rpm | | | pH |
| Jul. 28, 2021 | TE/10 rpm | TF/20 rpm | 10% | Aug. 11, 2021 | TE/10 rpm | TF/20 rpm | 10% |
| 272 000 | 164 000 | 98 500 | 8.19 | 348 000 | 207 000 | 117 000 | 8.25 |
| 210 000 | 126 000 | 87 500 | 8.07 | 286 000 | 174 000 | 97 500 | 8.20 |
| 278 000 | 169 000 | 113 000 | 8.22 | 362 000 | 213 000 | 125 000 | 8.66 |
| 248 000 | 151 000 | 99 500 | 8.31 | 338 000 | 194 000 | 116 000 | 8.40 |

| 28-days | | | | 38-days | | | |
|---|---|---|---|---|---|---|---|
| Viscosity, cps, 1 min | | | | Viscosity, cps, 1 min | | | |
| TE/5 rpm | | | pH | TE/5 rpm | | | pH |
| Aug. 18, 2021 | TE/10 rpm | TF/20 rpm | 10% | Aug. 30, 2021 | TE/10 rpm | TF/20 rpm | 10% |
| 352 000 | 202 000 | 119 000 | 8.16 | 358 000 | 227 000 | 130 000 | 8.37 |
| 278 000 | 169 000 | 103 000 | 8.24 | 284 000 | 171 000 | 112 000 | 8.23 |
| 364 000 | 211 000 | 136 000 | 8.30 | 366 000 | 225 000 | 145 000 | 8.92 |
| 282 000 | 195 000 | 121 000 | 8.52 | 326 000 | 198 000 | 126 000 | 8.70 |

| 68-days | | | |
|---|---|---|---|
| Viscosity, cps, 1 min | | | |
| TE/5 rpm | | | pH |
| Sep. 29, 2021 | TE/10 rpm | TF/20 rpm | 10% |
| 388 000 | 245 000 | 125 000 | 8.37 |
| 316 000 | 174 000 | 119 000 | 8.21 |
| 374 000 | 226 000 | 132 000 | 8.82 |
| 362 000 | 216 000 | 127 000 | 8.52 |

The viscosity and pH values were found to be within acceptable parameters for a dentifrice.

Next, the cleaning properties of these 4 dentifrices were evaluated using standard tests.

TABLE 11
Cleaning properties

| | CLEANING | | | |
|---|---|---|---|---|
| | PCR | RDA | PCR/RDA | REA |
| TCB | 76 | 90 | 0.84 | 4 |
| Treated oolitic aragonite | 76 | 87 | 0.87 | 5 |
| TCB + 0.5% bicarbonate | 81 | 95 | 0.85 | 4 |
| Treated oolitic aragonite + | 80 | 93 | 0.86 | 5 |
| 0.5% bicarbonate | | | | |

The pellicle cleaning ratio (PCR) test is used to evaluate the stain removal performance of oral care dentifrices such as toothpaste or toothpowders. All results were deemed acceptable. Relative dentin abrasivity (RDA) is a test for measuring the erosive effect of abrasives in toothpaste on tooth dentin. It involves using standardized abrasives compared against the test sample. The determination of this value is done by determining the activity while cleaning worn dentin which is radioactively marked by mild neutron irradiation. The values obtained depend on the size, quantity and surface structure of abrasive used in toothpastes. Values between 0 and 70 are representative of formulation having low dentin abrasivity, and values of 70-100 are representative of formulation having medium dentin abrasivity. The formulation tested were all deemed acceptable. Finally, the relative enamel abrasivity (REA) was determined. All results were deemed acceptable.

While preferred embodiments have been described above and illustrated in the accompanying drawings, it will be evident to those skilled in the art that modifications may be made without departing from this disclosure. Such modifications are considered as possible variants comprised in the scope of the disclosure.

1. An oral care composition comprising:
treated aragonite calcium carbonate (CaCO₃) particles having more than 95% (w/w) calcium carbonate content, a specific surface area (SSA) of about 2.70 to about 3.1 m²/g, for use as a first dental abrasive;
a fluoride compound suitable to provide beneficial fluoride treatment to teeth; and
a suitable carrier,
wherein said treated aragonite calcium carbonate particles have been effectively treated in mildly acidic condition to avoid reaction of fluoride from said fluoride compound and said treated aragonite calcium carbonate particles.

2. The oral care composition of claim 1, wherein said treated aragonite calcium carbonate particles have a particle size of from about 25 microns to about 70 microns, wherein said calcium carbonate content is from about 95% to about 99.9% (w/w); and/or wherein said particles have a specific surface area of from about 2.80 m²/g to about 2.9 m²/g.

3.-5. (canceled)

6. The oral care composition of claim 1, wherein said treated aragonite calcium carbonate (CaCO₃) particles are from an aragonite of vegetal origin.

7. The oral care composition of claim 6, wherein said aragonite of vegetal origin is oolittic aragonite.

8. The oral care composition of claim 1, wherein said fluoride compound is sodium fluoride (NaF), stannous fluoride (SnF₂), sodium monofluorophosphate (MFP), or combinations thereof.

9. The oral care composition of claim 1, wherein said fluoride compound provides a concentration of fluoride of from about 800 ppm to about 5000 ppm, or from about 1000 ppm to about 1500 ppm.

10. (canceled)

11. The oral care composition of claim 1, wherein said treated aragonite calcium carbonate particles is from about 0.100% to about 20% (w/w) of the composition.

12. The oral care composition of claim 1, wherein said particles have a crystallinity of about 24% to about 28%, or about 26%.

13. (canceled)

14. The oral composition of claim 1, further comprising a second dental abrasive, a thickening agent; a humectant, or combinations thereof.

15. The oral care composition of claim 14, wherein said second dental abrasive is a colloidal calcium, a colloidal silica, a hydrated silica, a sodium bicarbonate (NaHCO₃), aluminum hydroxide (Al(OH)₃), calcium carbonate (CaCO₃), a calcium hydrogen phosphate (CaHPO₄·2H₂O), an anhydrous calcium hydrogen phosphate, a silica, a zeolite, and hydroxyapatite (Ca₅(PO₄)₃OH), or a combination thereof;
wherein said thickening agent is a natural gum obtained from seaweeds: a natural gum obtained from non-marine botanical resource, a natural gum produced by bacterial fermentation, a starch, a pectin, a carboxymethyl cellulose, a hydroxypropyl cellulose, a methyl cellulose, a gelatin, a silica, or a combination thereof; and/or
wherein said humectant is selected from the group consisting of propylene glycol, hexylene glycol, butylene glycol, glyceryl triacetate, neoagarobiose, a sugar polyol, a polymeric polyol, quillaia, lactic acid, urea, glycerin, aloe vera gel, MP Diol, an alpha hydroxy acid, and honey.

16.-21. (canceled)

22. The oral care composition of claim 21, wherein said natural gums obtained from seaweeds is chosen from agar (E406), alginic acid (E400), Sodium alginate (E401), potassium alginate, ammonium alginate, calcium alginate, carrageenan (E407), or a combination thereof:
wherein said natural gum obtained from non-marine botanical resource is chosen from acacia gum, gum arabic (E414), gum ghatti, gum tragacanth (E413), karaya gum (E416), guar gum (E412), locust bean gum (E410), beta-glucan, chicle gum, dammar gum, Glucomannan (E425), mastic gum, psyllium seed husks, spruce gum, tara gum (E417), or a combination thereof; and
wherein said natural gum produced by bacterial fermentation is chosen from gellan gum (E418), Xanthan gum (E415), or a combination thereof.

23.-28. (canceled)

29. The oral care composition of claim 15, wherein said sugar polyol is chosen from glycerol, sorbitol, xylitol, maltitol, and a combination thereof:
wherein said polymeric polyol is polydextrose, polyethylene glycol, polypropylene glycol, poly(tetramethylene ether) glycol, and a combination thereof; and/or
wherein said alpha hydroxy acid is lactic acid.

30.-33. (canceled)

34. The oral care composition of claim 1, further comprising an emulsifier, a surfactant; a pH regulator, or combinations thereof.

35. The oral care composition of claim 34, wherein said emulsifier is lecithin, a vegetal pulp powder, a sodium citrate and citric acid, or a combination thereof:
wherein said surfactant is chosen from sodium lauryl sulfate, ammonium lauryl sulfate, sodium N-lauryl sarcosinate, sodium lauryl sulfoacetate, or a combination thereof; and/or
wherein said pH regulator is chosen from citric acid and its derivatives, phosphoric acid and its derivatives, trisodium phosphate, sodium citrate, lactic acid, bicarbonic acid, or a combination thereof.

36.-44. (canceled)

45. The oral composition of claim 1, further comprising a preservative, a solvent; and antimicrobial agent, or combinations thereof.

46. The oral composition of claim 45, wherein said preservative is chosen from a sorbitan sesquioleate derivative, sodium benzoate, benzoic acid, a eucalyptus extract, potassium sorbate, or a combination thereof:
wherein said solvent is chosen from water, ethanol, isopropanol, sorbitol and glycerol; and/or
wherein said antimicrobial agent is chosen from a natural essential oil, an antimicrobial phenolic compound, or a combination thereof.

47.-57. (canceled)

58. The oral composition of claim 1, wherein said treated aragonite calcium carbonate (CaCO₃) particles are free of chitin.

59.-60. (canceled)

61. A method of cleaning an oral cavity comprising applying the oral composition of claim 1 to an oral cavity.

62. A method of preventing formation of, or of removing calculus in an oral cavity comprising applying the oral composition of claim 1 to an oral cavity.

63.-79. (canceled)
